(19) United States
(12) Patent Application Publication
(10) Pub. No.: US 2025/0017544 A1
(43) Pub. Date: Jan. 16, 2025

(54) AUTOMATED DETERMINATION OF PEDIATRIC LOWER LIMB ALIGNMENT

(71) Applicant: The University of British Columbia, Vancouver (CA)

(72) Inventors: Anthony Cooper, Vancouver (CA); Yousif Murad, Vancouver (CA)

(21) Appl. No.: 18/773,341

(22) Filed: Jul. 15, 2024

Related U.S. Application Data
(60) Provisional application No. 63/526,752, filed on Jul. 14, 2023.

(51) Int. Cl. A61B 6/50; A61B 6/00; A61B 6/12; G06T 7/00; G06T 7/60; G06T 7/73; G06V 10/25; G06V 10/26; G06V 10/774; G06V 10/82

(52) U.S. Cl. CPC A61B 6/505; A61B 6/12; A61B 6/5217; G06T 7/0012; G06T 7/60; G06T 7/73; G06V 10/25; G06V 10/267; G06V 10/774; G06V 10/82; G06T 2207/10116; G06T 2207/20081; G06T 2207/20084; G06T 2207/30008; G06T 2207/30052; G06T 2207/30204; G06V 2201/033

(57) ABSTRACT

Systems and methods for performing a pediatric lower limb alignment assessment by: receiving a pediatric lower limb radiographic image; identifying a plurality of regions of interest in the radiographic image using a first artificial intelligence model such that each one of the plurality of regions of interest contains at least one of a plurality of anatomical features of interest; determining a plurality of landmark locations for each one of the plurality of identified regions of interest in the radiographic image using a second AI model; and calculating one or more parameter values representative of the pediatric lower limb alignment based on a geometric relationship between the plurality of landmark locations.

### CROSS-REFERENCE TO RELATED APPLICATIONS

This application claims priority to U.S. Provisional Patent Application No. 63/526,752, filed on Jul. 14, 2023, the entire contents of which is incorporated herein by reference for all purposes.

TECHNICAL FIELD

The present disclosure relates to automated determination of various anatomic characteristics and parameters for the lower limbs and in particular to the automated analysis of radiographic images to determine anatomic characteristics and parameters of the lower limbs using artificial intelligence (AI) models.

### BACKGROUND

Lower limb alignment is the quantification of a set of parameters that are commonly measured radiographically to test for and track a wide range of skeletal pathologies in that altered limb alignment is both a sign and cause of pathologies. However, determining limb alignment is a laborious task in the pediatric orthopedic setting.

In particular, alignment of the lower limbs is defined by the measurement of physiological axes in a given plane and comparing the results to population means. The physiological axes typically refer to the mechanical and anatomic axes of the femur and the tibia. The measurement of the axes and associated values can be achieved either clinically, as is often done initially, or by employing radiographic investigations. The primary tool for evaluating lower limb alignment is the anteroposterior (AP) standing radiograph, which can be analyzed to determine the measurement of physiological axes.

Specifically, the mechanical axis is often defined as a line connecting the centers of the femoral head and tibiotalar joint. The mechanical axis of the femur is often defined as a line connecting the centers of the femoral head and knee and mechanical axis of the tibia is a line connecting the centers of the knee and talus, tibial plafond or tibiotalar joint. The anatomical axis of the femur is often defined as a line connecting the center of the femoral shaft to a point 10 cm above the knee joint, equidistant between the medial and lateral cortices while the anatomical axis of the tibia is often defined as a line bisecting the midshaft tibia, coinciding with the mechanical axis of the tibia. The aforementioned axes define between them a range of angles important in clinical practice.

Measurements for lower limb alignment determination are usually performed manually. As such, this process is repetitive, arduous, and time-consuming. The measurements can also be prone to human error, technician inexperience, as well as lack of consistency and reproducibility.

Accordingly, systems and methods that enable automated analysis of radiographic images to determine anatomic measurements of the lower limbs remain highly desirable.

### SUMMARY

In accordance with one aspect of the present disclosure, a pediatric lower limb alignment assessment method is disclosed, the method comprising: receiving a pediatric lower limb radiographic image; identifying a plurality of regions of interest (ROIs) in the radiographic image using a first artificial intelligence (AI) model, each one of the plurality of ROIs containing at least one of a plurality of anatomical features of interest, each anatomical feature of interest comprising a respective portion of a bone; determining a plurality of landmark locations for each one of the plurality of identified ROIs in the radiographic image using a second AI model, each landmark location corresponding to a position within a respective anatomical feature of interest; and calculating at least one parameter value representative of the pediatric lower limb alignment based on a geometric relationship between the plurality of landmark locations.

In some aspects, the method further comprises: segmenting the radiographic image to generate a plurality of image segments using the first AI model, each one of the plurality of image segments corresponding to one of the plurality of ROIs, each one of the plurality of landmark locations determined from a respective one of the plurality of image segments by the second AI model.

In some aspects, the method further comprises: capturing the radiographic image.

In some aspects, the method further comprises: identifying a plurality of anatomical regions of interest using a third AI model, each anatomical region of interest comprising an entire bone; and determining the plurality of landmark locations using the plurality of anatomical features of interest and the plurality of anatomical regions of interest.

In some aspects, the second AI model is configured to identify the plurality of anatomical features of interest; and the plurality of landmark locations are determined using the plurality of anatomical features of interest.

In some aspects, the radiographic image is an anteroposterior standing weight-bearing radiograph.

In some aspects, the radiographic image includes hardware implants.

In some aspects, the method further comprises: obtaining radiographic images where at least one region of interest is identified; and training the first AI model using the obtained radiographic images to identify the at least one identified region of interest.

In some aspects, the method further comprises: obtaining image segments where each image segment corresponds to a respective region of interest; and training the first AI model using the obtained image segments to segment the radiographic image to generate a plurality of image segments based on the plurality of ROIs.

In some aspects, the method further comprises: obtaining radiographic images where at least one anatomical feature of interest is identified; and training the second AI model using the obtained radiographic images to identify the at least one identified anatomical feature of interest.

In some aspects, the method further comprises: obtaining radiographic images where at least one landmark location is identified; and training the second AI model using the obtained radiographic images to identify the at least one identified landmark location.

In some aspects, the method further comprises; obtaining radiographic images where at least one anatomical region of interest is identified; and training the third AI model using the obtained radiographic images to identify the at least one identified anatomical region of interest.

In some aspects, the plurality of ROIs and the plurality of anatomical features of interest comprise regions corresponding to: femoral head, greater trochanter, distal femur, proximal tibia, distal tibia, or combinations thereof.

In some aspects, the plurality of ROIs and the plurality of anatomical features of interest comprise a region corresponding to a radiopaque washer used as a size marker.

In some aspects, the first AI model and/or the second AI model is a residual neural network.

In some aspects, the first AI model comprises five convolutional neural networks (CNNs), each configured to identify a respective region of interest corresponding to one of: femoral head, greater trochanter, distal femur, proximal tibia, and distal tibia; and the first AI model comprises an additional convolutional neural network configured to identify a region of interest corresponding to a washer.

In some aspects, the second AI model comprises five convolutional neural networks (CNNs), each configured to identify a respective anatomical feature of interest corresponding to one of: femoral head, greater trochanter, distal femur, proximal tibia, and distal tibia; and the second AI model comprises an additional convolutional neural network configured to identify a feature of interest corresponding to a washer.

In some aspects, the at least one parameter value is at least one of: mechanical axes of the femur and tibia; a hip-knee angle; a mechanical lateral proximal femoral angle; a mechanical lateral distal femoral angle; a mechanical medial proximal tibial angle; a mechanical lateral distal tibial angle; a mechanical axis deviation; an anatomic medial proximal femoral angle; an anatomic lateral distal femora angle; an anatomic medial proximal tibial angle; an anatomic lateral distal tibial angle; an anatomic tibiofemoral angle; or a knee alignment.

In accordance with another aspect of the present disclosure, a method of determining pediatric lower limb alignment parameters is disclosed, the method comprising: receiving a pediatric lower limb radiograph; extracting regions of interest (ROIs) from the pediatric lower limb radiograph using a first set of convolutional neural networks (CNNs); segmenting bones in each extracted ROI using a second set of CNNs; segmenting bones in the full pediatric lower limb radiograph using a single, distinct CNN; identifying anatomic landmarks needed for alignment measurements on each of the segmented images; and calculating limb alignment parameters using the identified anatomic landmarks.

In some aspects, receiving a pediatric lower limb radiograph comprises receiving an anteroposterior standing weight-bearing radiograph.

In some aspects, extracting ROIs comprises extracting ROIs corresponding to the following: the femoral head, the greater trochanter, the distal femur, the proximal tibia, the distal tibia, and a radiopaque washer used as a size marker.

In some aspects, the CNN is residual neural network (ResNet).

In some aspects, the first set of CNNs comprises six CNNs.

In some aspects, the second set of CNNs comprises six CNNs.

In some aspects, segmenting bones in the full pediatric lower limb radiograph comprises segmenting the femur, tibia, and fibula.

In some aspects, identifying anatomic landmarks comprises identifying anatomic landmarks either manually, automatically or both.

In some aspects, calculating limb alignment parameters comprises calculating mechanical and anatomic axis angles.

In some aspects, calculating mechanical axis angles comprises calculating the following: the mechanical axes of the femur and tibia; the hip-knee angle; the mechanical lateral proximal femoral angle; the mechanical lateral distal femoral angle; the mechanical medial proximal tibial angle; the mechanical lateral distal tibial angle; and the mechanical axis deviation.

In some aspects, calculating anatomical axis angles comprises calculating the following: the anatomic medial proximal femoral angle; the anatomic lateral distal femoral angle; the anatomic medial proximal tibial angle; the anatomic lateral distal tibial angle; and the anatomic tibiofemoral angle.

In accordance with another aspect of the present disclosure, a method of training AI models for assessing pediatric lower limb alignment is disclosed, the method comprising: obtaining radiographic images where at least one region of interest is identified, each region of interest containing at least one of a plurality of anatomical features of interest, each anatomical feature of interest comprising a respective portion of a bone; obtaining radiographic images where at least one landmark location is identified, each landmark location corresponding to a position within a respective anatomical feature of interest; training a first AI model using the obtained radiographic images to identify the at least one identified region of interest using masks of the at least one region of interest as ground truth; and training the second AI model using the obtained radiographic images to identify the at least one identified landmark location using positions of the at least one landmark location as ground truth.

In some aspects, the method further comprises: obtaining image segments where each image segment corresponds to a respective region of interest; and training the first AI model using the obtained image segments to segment the radiographic image to generate a plurality of image segments based on the plurality of ROIs.

In some aspects, the method further comprises: obtaining radiographic images where at least one anatomical feature of interest is identified; and training the second AI model using the obtained radiographic images to identify the at least one identified anatomical feature of interest using masks of the at least one anatomical feature of interest as ground truth.

In some aspects, the method further comprises; obtaining radiographic images where at least one anatomical region of interest is identified, each anatomical region of interest comprising an entire bone; training a third AI model using the obtained radiographic images to identify the at least one identified anatomical region of interest using masks of the at least one anatomical region of interest as ground truth.

In some aspects, the plurality of ROIs and the plurality of anatomical features of interest comprise regions corresponding to: femoral head, greater trochanter, distal femur, proximal tibia, distal tibia, or combinations thereof.

In some aspects, the plurality of ROIs and the plurality of anatomical features of interest comprise a region corresponding to a radiopaque washer used as a size marker.

In some aspects, one or more of the first AI model, the second AI model, and the third AI model is a residual neural network.

In some aspects, the first AI model comprises five convolutional neural networks (CNNs), each configured to identify a respective region of interest corresponding to one of: femoral head, greater trochanter, distal femur, proximal tibia, and distal tibia; and the first AI model comprises an additional convolutional neural network configured to identify a region of interest corresponding to a washer.

In some aspects, the second AI model comprises five convolutional neural networks (CNNs), each configured to identify a respective anatomical feature of interest corresponding to one of: femoral head, greater trochanter, distal femur, proximal tibia, and distal tibia; and the second AI model comprises an additional convolutional neural network configured to identify a feature of interest corresponding to a washer.

In some aspects, one or more of the first AI model, the second AI model, and the third AI model is trained for provided outputs for calculating the one or more parameter values for assessing pediatric lower limb alignment.

In accordance with another aspect of the present disclosure, a system for determining a parameter value of lower limb alignment is disclose, the system comprising one or more processing units configured to perform the method of any one of the above aspects.

In accordance with another aspect of the present disclosure, a non-transitory computer-readable medium having computer readable instructions stored thereon is disclosed, which, when executed by one or more processing units, causes the one or more processing units to perform the method of any one of the above aspects.

This summary does not necessarily describe the entire scope of all aspects. Other aspects, features, and advantages will be apparent to those of ordinary skill in the art upon review of the following description of specific embodiments.

### BRIEF DESCRIPTION OF THE DRAWINGS

Further features and advantages of the present disclosure will become apparent from the following detailed description, taken in combination with the appended drawings, in which:

FIG. 1 depicts a system for automatic analysis of lower limb characteristics from radiographic images, according to an example embodiment.

FIG. 2 depicts a process for the automatic analysis of lower limb characteristics from radiographic images utilized by the system of FIG. 1, according to an example embodiment.

FIG. 3 depicts an example initial input for the process of FIG. 2 as well as corresponding example outputs at various stages of the process according to FIG. 2.

FIG. 4 depicts a method for automatic analysis of lower limb characteristics from radiographic images utilized by the process of FIG. 2.

FIGS. 5A-5C respectively depict a process for training a first, second, and third AI model utilized by the process of FIG. 2 to automatically analyze lower limb characteristics from radiographic images, according to example embodiments.

FIG. 6 depicts a method for training the first and second AI models according to the processes of FIGS. 5A-5C.

FIG. 7 depicts a graphical user interface of the system of FIG. 1, according to an example embodiment.

FIG. 8A depicts example results showing regions of interest (ROIs) identified by the process of FIG. 2.

FIG. 8B depicts example results showing anatomical features of interest identification by the process of FIG. 2.

FIG. 8C depicts example results showing full anatomical features identified by the process of FIG. 2.

FIG. 9 depicts example performance results of the process of FIG. 2 in analysis of lower limb characteristics.

FIG. 10 depicts example performance results of the process of FIG. 2 in determination of lower limb characteristics from radiographic images.

FIG. 11 depicts example lower limb characteristics determined by the process of FIG. 2 as tracked for single-cases.

FIG. 12 depicts example lower limb characteristics determined by the process of FIG. 2 in comparison to lower limb characteristics determined manually.

FIG. 13 depicts the distribution of performance scores for example lower limb anatomical features determined by the process of FIG. 2.

It will be noted that throughout the appended drawings, like features are identified by like reference numerals.

### DETAILED DESCRIPTION

The determination of lower limb alignment by the characterization of physiological axes and calculations of the associated characteristic values is a commonly performed process, both in surgical practice and in research. Clinically, lower limb alignment is mostly determined manually. However, the repetitive and time-consuming nature of this task lends itself well as a target for automation. Automation can serve to improve the reliability and reproducibility of the measurements. Semi-automated as well as fully automated methods for lower limb analysis can be used to perform measurements quickly and accurately, but none have been shown to provide a full interpretation of the lower limb axes to calculate the characteristic values.

Accordingly, the present disclosure provides herein an artificial intelligence (AI) based approach for determining lower limb alignment and characteristic values. Specifically, the AI model may include at least one convolutional neural network (CNN) to provide a fully automated workflow in measuring lower limb alignment. CNNs are a deep learning architecture that is inspired by natural visual perception mechanisms and can be applied to various areas such as image classification, segmentation and pattern recognition. The CNNs may be utilized in a machine learning approach to segment pediatric weight-bearing lower limb radiographs. Anatomic landmark features may be extracted from the output of the CNNs with which lower limb alignment parameters can be calculated using geometric relationships in a computationally efficient manner.

In accordance with the present disclosure, systems and method for the automatic determination of characteristic values of lower limb alignment, and in particular pediatric lower limb alignment, are disclosed. The systems and methods disclosed herein can be used to analyze a radiographic image and return the calculated characteristic values of lower limb alignment for the given radiographic image. The radiographic image is received and processed by a first AI model to identify one or more regions of interest (ROIs). The radiographic image can be segmented according to the identified ROIs. A second AI model can identify one or more anatomical features of interest using the identified ROIs, which can be used to determine the locations of the landmark features (e.g. landmark locations) required to calculate the characteristic values for lower limb alignment. By using the positions of the landmark features relative to one another, geometric calculations (e.g. distance between two points, angle of three points, angle between two vectors) can be performed to determine the characteristic values for lower limb alignment. Systems and methods for training the AI models are also disclosed.

Advantageously, the systems and methods of the present disclosure can allow for quick and computationally efficient calculations of different characteristic values for lower limb alignment. The embodiments described herein may be able to calculate a full set of characteristic values (as described further herein) within two seconds. Beyond indicating the characteristic values to be calculated, manual actions may not be required. The radiographic image to be analyzed can be unmodified and input “as-is”. Further, the systems and methods of the present disclosure can also process radiographic images that include hardware implants. As will be apparent in the present disclosure, the systems and methods of the present disclosure may automatically calculate characteristic values for lower limb alignment quickly and efficiently.

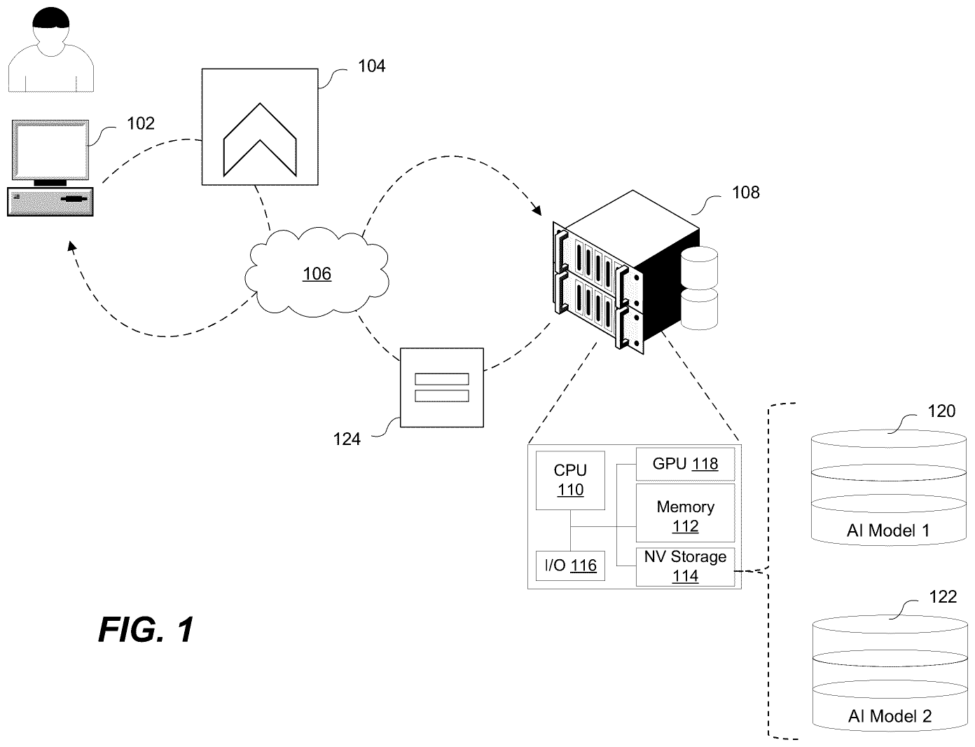

Embodiments are described below, by way of example only, with reference to FIGS. 1-13.

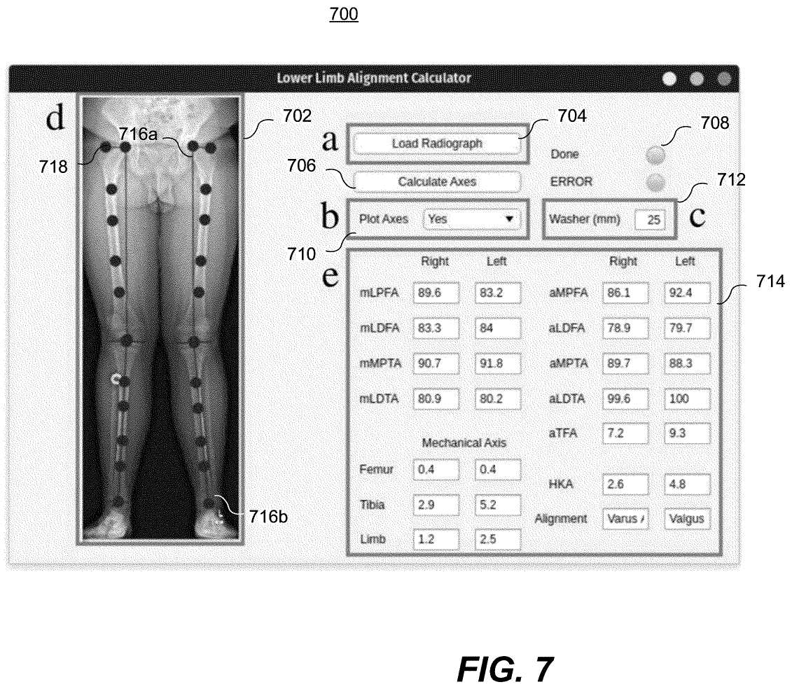

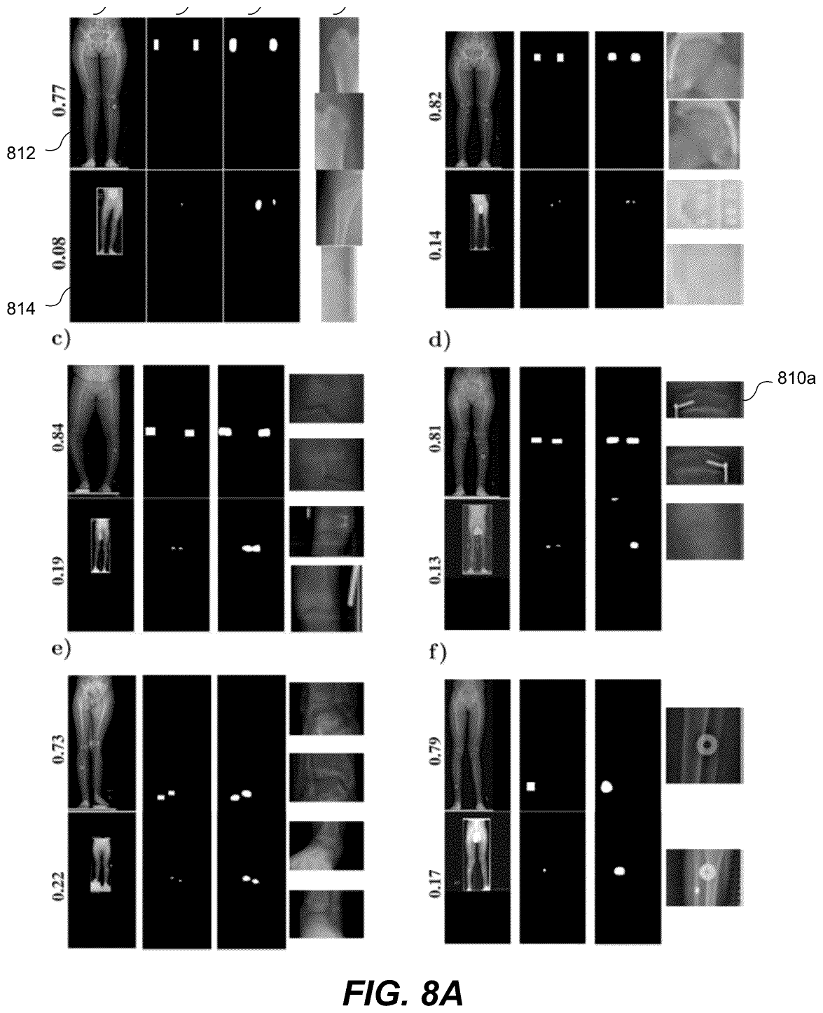

FIG. 1 depicts a system for automatic analysis for lower limb characteristics from radiographic images, according to an example embodiment, shown in FIG. 1 as one or more servers 108. The implementation of the servers 108 is not restrictive and servers 108 may be a physical server, cloud-based server, or a hybrid thereof, for example. An user may interact with the servers 108 via a device 102 over a communications network 102 (e.g. the internet). The device 102 may be a computer, as depicted in FIG. 1, but is not restricted to those expressly shown and may be any suitable device known in the art such as smart phones and tablets. The servers 108 may provide a graphical user interface (GUI) on the device 102 for ease of communication and operation control by the user. The implementation of the GUI is not restrictive and may be, for example, a mobile/computer application or a web page. An example GUI implemented for interaction with the servers 108 is shown and described in further detail with respect to FIG. 7. The GUI can be used to provide input to and receive output from the servers 108.

According to the present disclosure, a radiographic image 104 may be provided to the servers 108, for example, from the device 102. The radiographic image 104 may be an anteroposterior radiograph and in particular an anteroposterior standing weight-bearing radiograph. The radiographic image 104 may be in a standard image format such as JPEG or PNG. The servers 108 are configured to analyze the radiographic image 104 and calculate one or more characteristic values 124 for lower limb alignment from the radiographic image 104. The servers 108 may also determine a type of lower limb alignment (e.g. normal alignment, varus alignment, or valgus alignment) from the calculated characteristic values. That is, the servers 108 may automatically calculate the one or more characteristic values 124 for the lower limbs of the radiographic image 104. The characteristic values 104 may include one or more of: mechanical lateral proximal femoral angle (mLPFA), mechanical lateral distal femoral angle (mLDFA), mechanical medial proximal tibial angle (mMPTA), mechanical lateral distal tibial angle (mLDTA), anatomic medial proximal femoral angle (aMPFA), anatomic lateral distal femoral angle (aLDFA), anatomic medial proximal tibial angle (aMPTA), anatomic lateral distal tibial angle (aLDTA), anatomic tibiofemoral angle (aTFA), hip-knee-ankle angle (HKA), or mechanical axis deviation (MAD). Further, the lower limb alignment type may be determined from the characteristic values, for example, from the calculated HKA. It should be noted that the above values and lower limb alignment type may be calculated for the right and/or left lower limb. The servers 108 may receive a selection of values to be calculated, for example from the GUI provided on the device 102. Alternatively, the servers 108 may calculate all of the above values without any selection or input from the device 102. As depicted in FIG. 1, the calculated values 124 are returned and provided to the device 102.

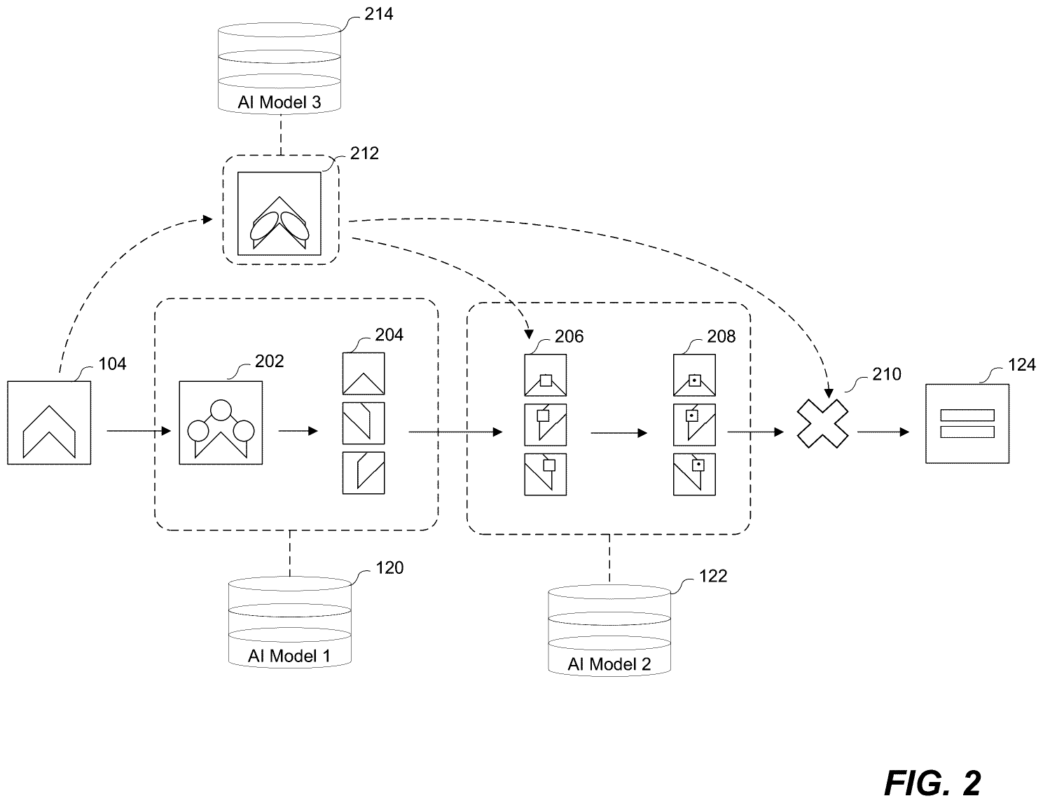

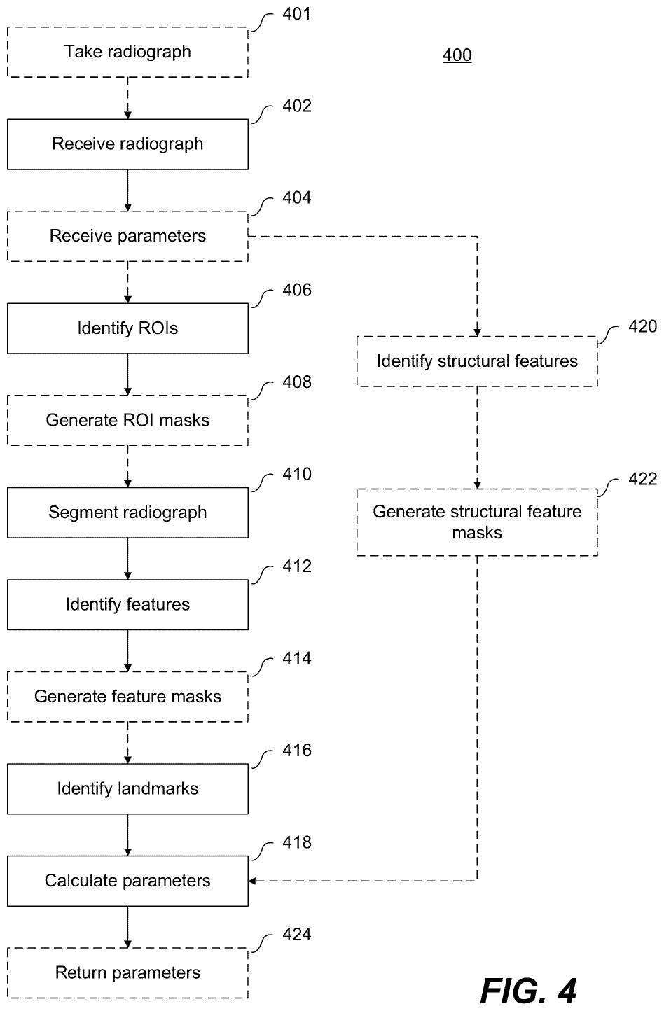

To calculate the characteristic values 124, the servers 108 may identify one or more ROIs corresponding to one or more anatomical features using a first AI model 120. The first AI model 120 may segment the radiographic image 104 to generate a plurality of segmented images, each corresponding to at least one specific region of interest (ROI). For example, the segmented images may include cropped radiographic images corresponding to regions corresponding to one or more of: femoral head, greater trochanter, distal femur, distal tibia, proximal tibia, or washer (e.g. a scale marker). The segmented radiographic images may be used as input for a second AI model 122 to identify anatomical features corresponding to the ROIs. The identified anatomical features may be one or more of: femoral head, greater trochanter, distal femur, distal tibia, proximal tibia, or washer. By identifying the one or more anatomical features, the second AI model 122 can identify one or more landmark features or locations using the identified anatomical features as reference. The landmark features can be used to calculate the characteristic values, as needed. In particular, by using the positions (e.g. x and y values within the radiographic image) of the landmark locations, it is possible to calculate the characteristic values by establishing positional relativity between relevant landmark locations to calculate the corresponding characteristic values using geometric relationships/trigonometry. For example, the angle between points A, B, and C can be calculated using trigonometry equations if the positions of the points are known. The process for the automatic calculations of the characteristic values 124 from the radiographic image 104 is described in more detail herein with reference to FIG. 2. The corresponding method is described in further detail herein with reference to FIG. 4.

In a particular implementation, the servers 108 each comprise a CPU 110, a non-transitory computer-readable memory 112, a non-volatile storage 114, an input/output interface 116, and graphical processing units (“GPU”) 118. The non-transitory computer-readable memory 112 comprises computer-executable instructions stored thereon at runtime which, when executed by the CPU 110, configure the server to perform the above described processes of automatic characteristic value calculation. The non-volatile storage 114 has stored on it computer-executable instructions that are loaded into the non-transitory computer-readable memory 112 at runtime. The input/output interface 116 allows the server to communicate with one or more external devices such the device 102 (e.g. via network 106). The non-transitory computer-readable memory 112 may also have thereon the first AI model 120 and the second AI model 122. The GPU 118 may be used to control a display and may be used process the radiographic image 104 and to identify the ROIs, anatomical features, and landmark locations. In some aspects, the first AI model 120 and the second AI model 122may be stored at one or more separate servers. The CPU 110 and GPU 118 may be one or more processors or microprocessors, which are examples of suitable processing units, which may additional or alternatively comprise an artificial intelligence accelerator, programmable logic controller, a microcontroller (which comprises both a processing unit and a non-transitory computer readable medium), AI accelerator, neural processing unit (NPU), or system-on-a-chip (SoC). As an alternative to an implementation that relies on processor-executed computer program code, a hardware-based implementation may be used. For example, an application-specific integrated circuit (ASIC), field programmable gate array (FPGA), or other suitable type of hardware implementation may be used as an alternative to or to supplement an implementation that relies primarily on a processor executing computer program code stored on a computer medium.

It should be noted that while FIG. 1 depicts the device 102 and the servers 108 as separate entities coupled over the communication network 106, the device 102 and servers 108 may also be coupled directly/physically using cable(s) for data transfer. In some embodiments, the servers 108 may also be the device 102 or comprise the device 102 (e.g. the servers 108 being implemented as a part of a computer system). In such an embodiment, the servers 108 may directly retrieve the radiographic image 104 from local storage or removable local storage. In some embodiments, the device 102 and/or the servers 108 may be coupled to an instrument/device that is configured to capture the radiographic image 104 such that the radiographic image 104 can be received directly from the instrument/device once captured.

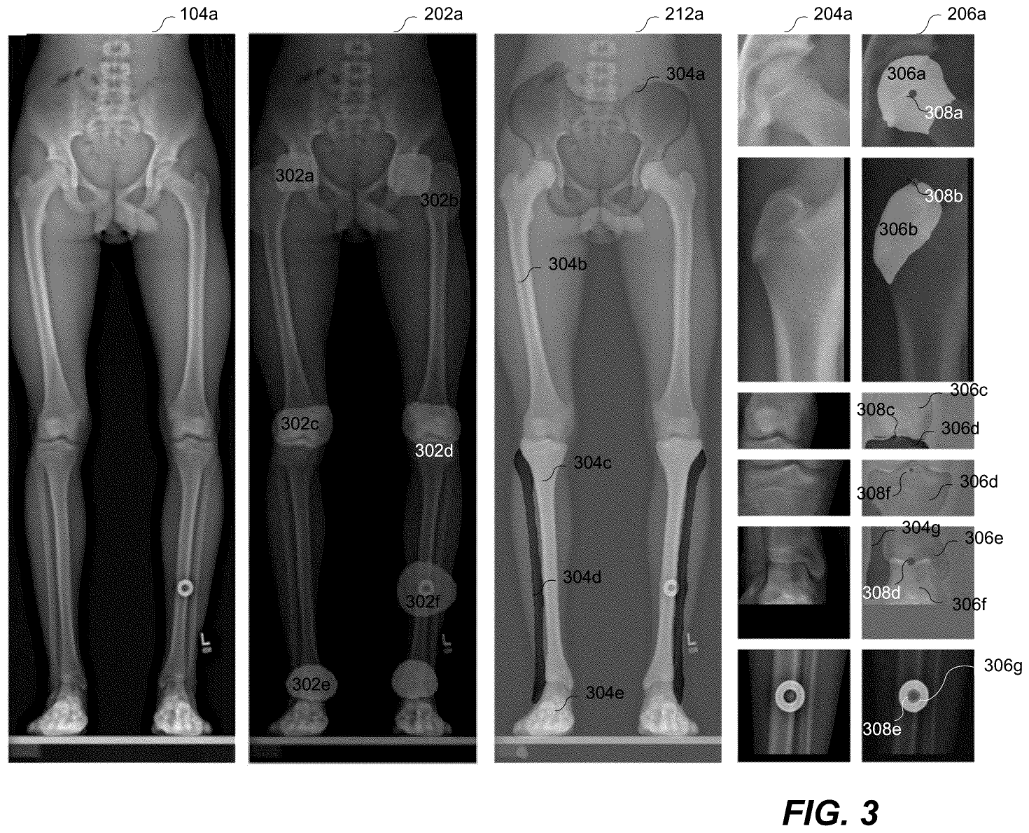

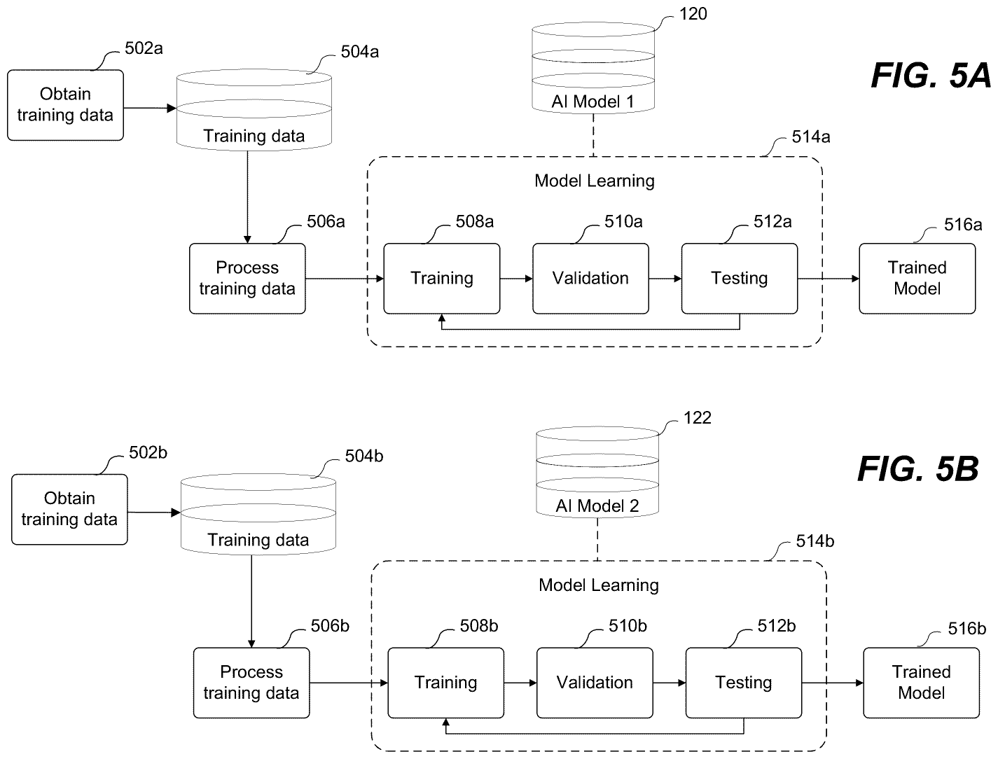

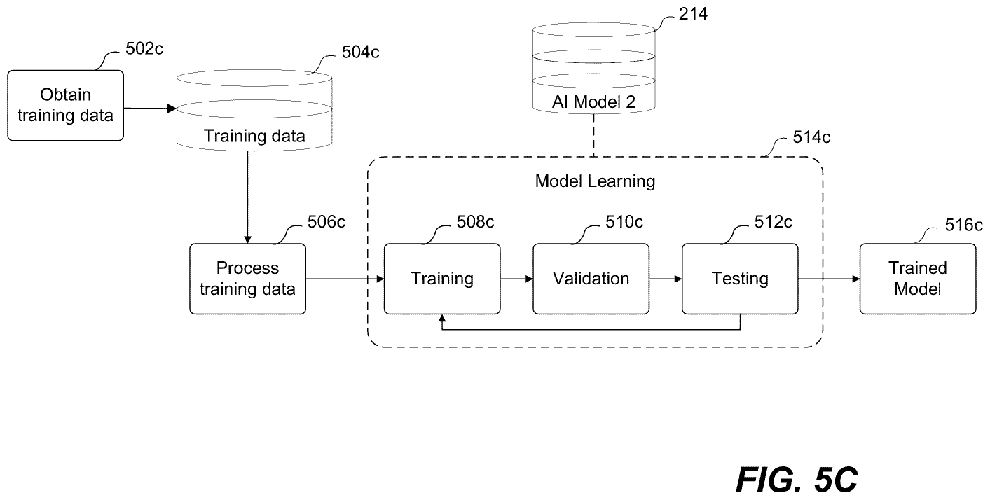

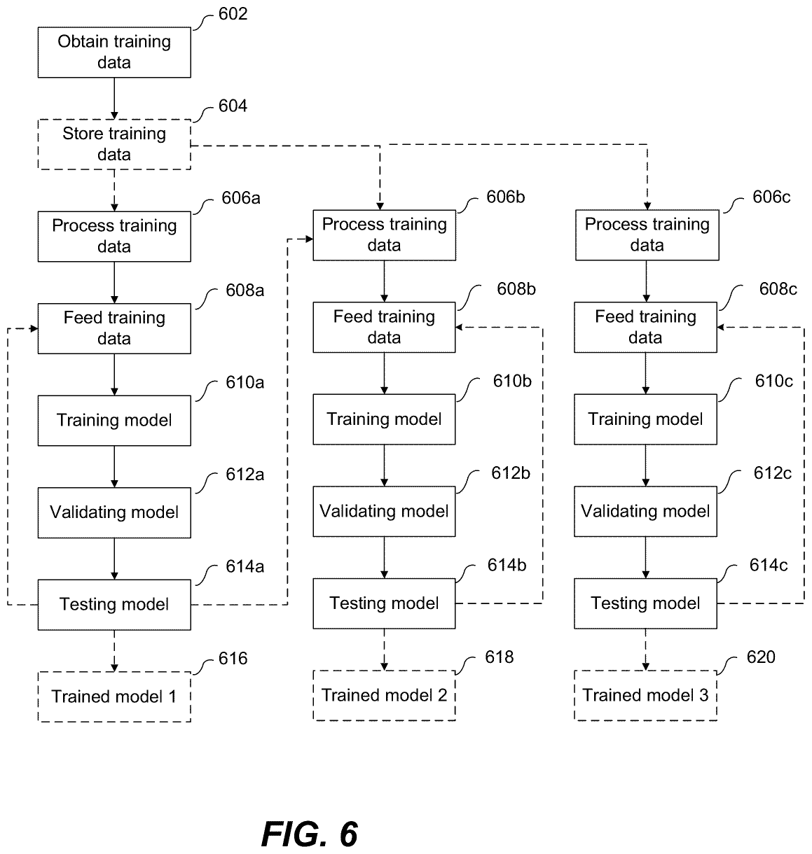

FIG. 2 depicts a process for automatic analysis of lower limb characteristics from radiographic images utilized by the servers 108 of FIG. 1, according to an example embodiment, which is described herein with reference to FIG. 3, where FIG. 3 depicts the example output result(s) of by the servers 108 at various stage of the analysis. As depicted in FIG. 2, the radiographic image 104 received by the servers 108 can be provided to the first AI model 120 as input. In some embodiments, the radiographic image 104 may be resized to meet a certain pre-set standard to ensure consistency. Alternatively, zero padding scaling may be done to scale the radiographic image 104 to the standard image size. The first AI model 120 is configured to identify one or more ROIs (202) from the radiographic image 104. The first AI model 120 may be a CNN based AI model that is trained to identify one or more ROIs. The training of the first AI model 120 is described in detail with reference to FIG. 5A and FIG. 6. The first AI model 120 can analyze the input radiographic image 104 to identify one or more ROIs in the radiographic image 104. For example, a radiographic image 104a (as shown in FIG. 3) can be provided to the first AI model 120. A plurality of ROIs 302a, 302b, 302c, 302d, 302e, and 302f are identified from the radiographic image 104a, which are shown as highlighted regions in the highlighted radiographic image 202a. As shown in the highlighted radiographic image 202a, the ROIs can be identified for both lower limbs. It should be also noted that fewer ROIs may be identified, for example if the characteristic values to be calculated do not require the identification of one or more of the ROIs. Each of the ROIs identified (e.g. highlighted) by the first AI model 120 may indicate or correspond to a particular anatomic or landmark feature of interest. That is, the first AI model 120 can determine a general region where a certain anatomical feature of interest could be located. For example, each of the highlighted ROIs 302a, 302b, 302c, 302d, 302e, and 302f can indicate that a particular anatomical feature of interest is located within the highlighted area. As such, each of the identified ROIs 302a, 302b, 302c, 302d, 302e, and 302f identifies a general location/region for a particular anatomical feature of interest.

In some embodiments, the first AI model 120 may identify ROIs corresponding to the locations of anatomical features of interest and more specifically to one or more of: femoral head, greater trochanter, distal femur, proximal tibia, distal tibia, or washer. That is, each ROI may be a general region of the radiographic image 104 that comprises a respective anatomical feature of interest. Each anatomical feature of interest may comprise a respective portion of a bone (or a region occupied by the respective portion of the bone). Specifically, as depicted in FIG. 3, the ROI 302a may identify a region corresponding to the femoral head; the ROI 302b may identify a region corresponding to the greater trochanter; the ROI 302c may identify a region corresponding to the distal femur; the ROI 302d may identify a region corresponding to the proximal tibia; the ROI 302e may identify a region corresponding to the distal tibia; and the ROI 302f may a identify region corresponding to a washer used as a size marker. The washer marker may be a radiopaque used a calibration marker and can have a known size, which may be included to facilitate length/distance measurements/calculations by using the known size as a scale (e.g. by correlating a physical length to a length in pixels). It should be noted that each ROI may comprise more than one anatomical feature and/or anatomical features not identified above but are associated with or in close proximity to the above identified anatomical features. Although FIG. 3 depicts that the ROIs are identified as highlighted regions, the ROIs may also be marked using a box or borders of other various shapes. In some embodiments, the ROIs may not be visually indicated; instead, data (e.g. coordinates) that would indicate the regions corresponding to the ROIs may be output by the first AI model 120. In particular, each of the ROIs may be output as or identified using a mask.

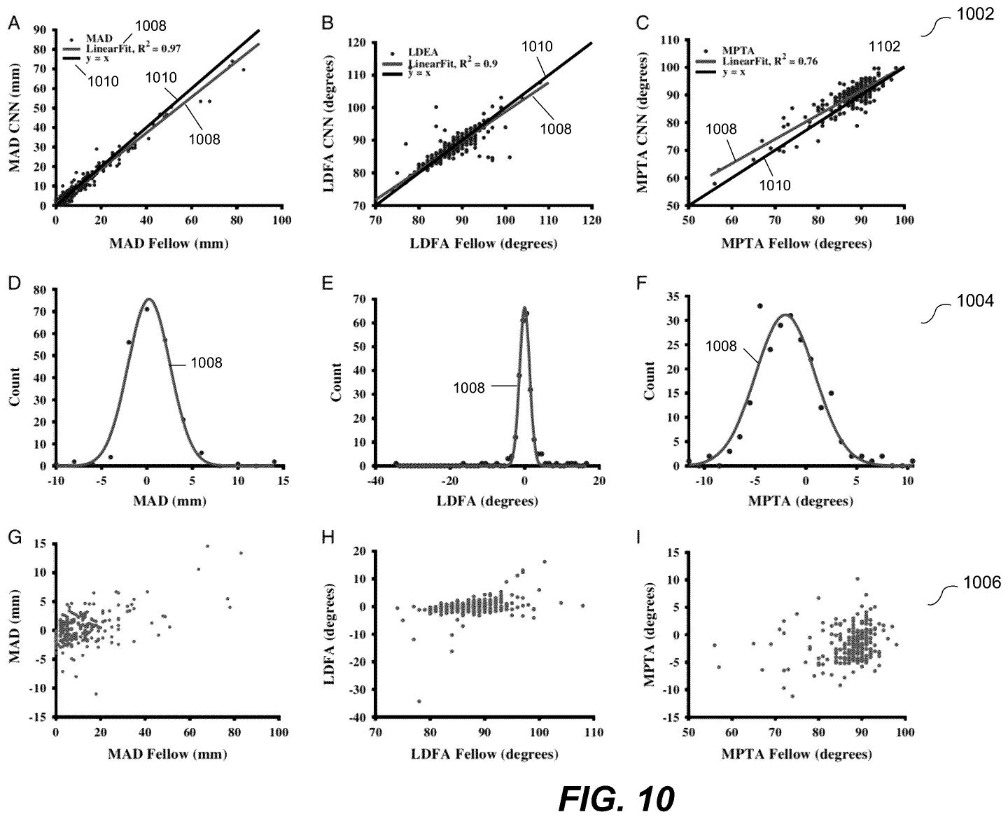

According to a particular implementation of the present disclosure, the first AI model 120 may be a set of 6 CNNs connected in parallel to receive and each independently process radiographic image(s). The CNNs may be residual network (ResNet) CNNs and in particular may have 50 layers. Specifically, each of the 6 CNNs may be configured to determine a particular ROI. That is, each of the CNNs can respective identify a ROI corresponding to one of: femoral head, greater trochanter, distal femur, proximal tibia, distal tibia, and washer. The input image may be a size of 512 by 256 pixels. The effectiveness and results of this particular implementation is described in further detail herein with respect to FIGS. 10-13.

Referring back to FIG. 2, the first AI model 120 can segment the radiographic image 104 to generate a plurality of segmented images (204), which may be a cropped portion of the radiographic image 104. Each segmented image may correspond to or comprise a particular ROI or ROIs. In particular, as shown in FIG. 3, the first AI model 120 may generate segmented images 204a corresponding to ROIs identifying one or more of: femoral head, greater trochanter, distal femur, proximal tibia, distal tibia, or washer. As depicted in FIG. 3 each segmented image corresponds to a particular ROI which identifies one of the anatomical features of interest (i.e. femoral head, greater trochanter, distal femur, proximal tibia, distal tibia, or washer). It should be noted that due to the proximity of certain anatomical features (e.g. the distal femur and proximal tibia), the identified ROI of a particular anatomical feature may overlap with another ROI of a different anatomical feature. Similarly, the segmented images may comprise more than one ROI and by extension more than one anatomical feature. For example, as shown in the segmented images 204a, the distal femur and proximal tibia may be shown in the same segmented image. In some embodiments, the radiographic image 104 may be segmented based on one or more ROIs or the intersection/overlap of two or more ROIs. For example, a segmented image may be specifically segmented such that the connection of the distal femur and proximal tibia is shown. In some embodiments, the radiographic image 104 may be segmented manually based on the ROIs identified by the first AI model 120. The image segmentation may also be performed for the ROIs in both lower limbs. It should be noted that each segmented image may contain or be associated with data that indicate the position (e.g. coordinates in x and y) of the segmented image relative to the original radiographic image 104 such that the position of any items identified in the segmented image relative to the original radiographic image 104 can be calculated.

According to a particular implementation, the first AI model 120 may generate 6 segmented images, each corresponding to a respective ROI containing one of femoral head, greater trochanter, distal femur, proximal tibia, distal tibia, and washer. The segmented images may be output by the first AI model 120. In particular, the respective sizes of the segmented image corresponding to the femoral head, greater trochanter, distal femur, proximal tibia, distal tibia, and washer may be: 256pixels by 256 pixels, 512 pixels by 256 pixels, 256 pixels by 512 pixels, 256 pixels by 256 pixels, 256 pixels by 512 pixels, and 256 pixels by 256 pixels.

Referring back to FIG. 2, the second AI model 122 can identify the anatomical features of interest from the segmented images (e.g. output by the first AI model 120), which can be performed for both limbs. For example, the segmented images output from the first AI model 120 may be used as input for the second AI model 122, as shown in FIG. 2. The second AI model 122 may be a CNN based AI model that is trained to identify one or more anatomical features of interest. The training of the second AI model 122 is described in detail with reference to FIG. 5B and FIG. 6. The second AI model 122 may identify at least one anatomical feature from each segmented image directly or in combination with the corresponding ROIs identified by the first AI model 120. That is, for each segmented image, the second AI model 122 may be configured to identify the at least one anatomical feature contained in the segmented image. Specifically, the second AI model 122 may be configured to identify anatomical features of interest in a particular area (i.e. the segmented image) The anatomical feature(s) can be identified/shown on the segmented image, as shown in processed images 206a in FIG. 3, on each of which at least one anatomical feature is identified. More generally, each anatomical feature of interest can comprise a respective bone and may identify the specific region in the segmented image that is occupied by the respective portion of the bone. This differs from the identified ROIs in that the ROIs may more generally define regions containing anatomical features of interest while the anatomical features of interest may more precisely identity the corresponding region. Specifically, the second AI model 122 may identify anatomical features of interest being at least one of: femoral head, greater trochanter, distal femur, proximal tibia, distal tibia, foot (e.g. tibiotalar and/or talus), fibula, and washer. As depicted in FIG. 3, the second AI model 122 may determine: a region 306a corresponding to the femoral head; a region 306b corresponding to the greater trochanter; a region 306c corresponding to the distal femur; a region 306d corresponding to the proximal tibia; a region 306e corresponding to the distal tibia; and a region 306e corresponding to the washer. In some embodiments, the second AI model 122 may also identify a region 306f corresponding to the foot (e.g. tibiotalar and/or talus) and/or a region 306g corresponding to the fibula.

As shown in FIG. 3, each processed image 206a corresponds to an anatomical feature of interest, which is identified on the segmented image, although more than one such feature may be identified and included in the same segmented image. Although FIG. 3 depicts the anatomical features as highlighted regions, the ROIs may also be marked/identified in other ways such as by means of borders. In some embodiments, the anatomical features may not be visually indicated, instead, data (e.g. coordinates in x and y) that would indicate the regions corresponding to the anatomical features may be output by the second AI model 122. In particular, each of the identified features may be output as or identified using a mask. It should be noted that it is possible for anatomical features to overlap with one another in the segmented radiographic image. As such, the anatomical features identified by the second AI model 122 may also overlap with one another.

Referring back to FIG. 2, the second AI model 122 may also identify one or more landmark locations, which can be performed for both limbs. For example, the previously identified one or more anatomical features of interest may be used as a basis to determine the one or more landmark locations. The landmark locations may also be determined directly from the segmented images and the identified ROIs. Accordingly, the second AI model 122 may also be configured and trained to identify one or more landmark locations. The training of the second AI model 122 for identifying the landmark locations is described in detail with reference to FIG. 5B and FIG. 6.

The second AI model 122 may identify one landmark location from each segmented image, where each of the landmark locations may be identified based on the anatomical feature or features of interest in the corresponding segmented image. The landmark locations can be identified/shown on the segmented image, shown as dots in processed images 206a of FIG. 3. Specifically, the second AI model 122 may identify landmark locations corresponding to: center of the femoral head, top of the greater trochanter (e.g. superior-lateral margin) corresponding to the center of the femoral shaft, upper knee center (e.g. on a line tangent to the femoral condyles), lower knee center (e.g. on a line tangent to the tibial plateau), center of the tibiotalar joint, or the center of the washer. As depicted in FIG. 3, the second AI model 122 may determine the landmark locations of: center of the femoral head 308a, top of the greater trochanter corresponding to the center of the femoral shaft 308b, upper knee center 308c, lower knee center 308f, tibiotalar joint 308d, and the center of the washer 308e. it should be noted that each of the landmark locations are determined using the positions of the corresponding anatomical feature of interest or the positions of multiple anatomical features of interest. For example, the center of the femoral head 308a can be identified by calculating the center of the region 306a corresponding to the femoral head and the upper knee center 308c can be determined by calculating the central point between the local minima of the region 306c corresponding to the distal femur (e.g. the lowest points on the femoral condyles). That is, each landmark location may be a position within a respective identified anatomical feature of interest and determined based on the respective anatomical feature of interest or multiple anatomical features of interest. Although FIG. 3 depicts the landmark locations visually as highlighted dots on the segmented radiographic images, the landmark locations may not be visually indicated; instead, data (e.g. coordinates in x and y) that would indicate the locations of the landmark locations may be output by the second AI model 122. In some embodiments, the landmark locations may be identified manually, or a combination of the second AI model 122 and manual identification may be performed to identify the landmark locations. Alternatively, some of landmarks locations may be determined manually with the remainder determined by the second AI model 122.

According to a particular implementation of the present disclosure, the second AI model 122 may be a set of 6 CNNs. The CNNs may be residual network (ResNet) CNNs and in particular may have 50 layers. Specifically, each of the 6 CNN may be configured to determine a particular anatomical feature of interest or multiple anatomical features of interest. That is, each of the CNNs can respective identify an anatomical feature corresponding to one or more of femoral head, greater trochanter, distal femur, proximal tibia, distal tibia, or washer. The second AI model 122 may generate 6 augmented segmented images, each corresponding to a respective one or more anatomical features being one ore more of femoral head, greater trochanter, distal femur, proximal tibia, distal tibia, washer, foot, or fibula. In particular, the respective sizes of the augmented segmented image corresponding to the femoral head, greater trochanter, distal femur, proximal tibia, distal tibia, and washer may be:

256 pixels by 256 pixels, 512 pixels by 256 pixels, 256 pixels by 512 pixels, 256 pixels by 256 pixels, 256 pixels by 512 pixels, and 256 pixels by 256 pixels, which are the same as the input images. Each of the CNNs may also be configured to determine a specific landmark location being: center of the femoral head, top of the greater trochanter corresponding to the center of the femoral shaft, upper knee center, lower knee center, center of the tibiotalar joint, or center of the washer. The landmark locations can be respectively included in the augmented segmented images and may be associated with: femoral head (for the center of the femoral head), greater trochanter (for the top of the greater trochanter corresponding to the center of the femoral shaft), distal femur (for the upper knee center), proximal tibia (for lower knee center), distal tibia (for the center of the tibiotalar joint), or washer (for the center of the washer). The effectiveness and results of this particular implementation is described in further detail herein with respect to FIGS. 10-13.

Referring back to FIG. 2, the landmark locations determined by the second AI model 122 may be used to calculate one or more characteristic values of lower limb alignment (210), which may be performed for both limbs. In particular, by using the positions of the landmark locations, it is possible to determine/calculate/plot the physiological axes by relating the positions/coordinates of the landmark locations to each other. For example, the mechanical axis (of the limb) can be identified using a line connecting the center of the femoral head 308a and the center of the tibiotalar joint 308d; the mechanical axis of the femur can be identified using a line connecting the center of the femoral head 308a and the upper knee center 308c; mechanical axis for the tibia (and the anatomical axis) can be identified using a line connecting the lower knee center 308f and the center of the tibiotalar joint 308d; the anatomical axis of the femur can be identified using a line connecting the upper knee center 308c and the top of the greater trochanter corresponding to the center of the femoral shaft 308b; and the anatomical axis of the tibia can be identified using a line that bisects the tibial shaft 306d. A line between the top of the greater trochanter corresponding to the center of the femoral shaft 308b and the center femoral head 308a can also be identified. In some embodiments, an a machine learning model (e.g. the second AI model 122) or data analysis algorithm may also determine an upper/lower joint line of the knee and/or a joint line of the foot (e.g. a line parallel to the distal tibial plafond) using the identified anatomical features of interest. For example, the upper joint line of the knee may be determined using the local minima of the region 306c corresponding to the distal femur, which can be a line tangent to the femoral condyles (e.g. knee base line) and the lower joint line of the knee may be determined using the local minima at the top of the region 306d corresponding to the proximal tibia, which can be a line tangent to the tibial plateau. The identified axes/lines may be used in the calculations of the characteristic values. It would be appreciated that other axes/lines may be identified using the landmark locations for use in the calculations of the characteristic values.

Possible definitions for the characteristic values calculated may be as follows: mLPFA: angle between the mechanical axis of the femur and the line between the top of the greater trochanter corresponding to the center of the femoral shaft and the center of the femoral head; mLDFA: angle between the mechanical axis of the femur and the upper joint line of the knee; mMPTA: angle between the mechanical axis of the tibia and the lower joint line of the knee; mLDTA: angle between the mechanical axis of the tibia and the joint line of the foot; aMPFA: angle between the anatomical axis of the femur and the line between the top of the greater trochanter corresponding to the center of the femoral shaft and the center of the femoral head; aLDFA: angle between the anatomical axis of the femur and the upper joint line of the knee; aMPTA: angle between the anatomical axis of the tibia and the lower joint line of the knee; aLDTA: angle between the anatomical axis of the tibia and the joint line of the foot; aTFA: angle between the anatomical axis of the femur and the anatomical axis of the tibia; HKA: angle between the mechanical axis of the femur and the anatomical axis of the tibia; and MAD: distance between the upper knee center and the mechanical axis of the limb. It would be appreciated that each line/axis can also be defined as (the connection between) two points, as known in linear algebra and vector mathematics.

Accordingly, trigonometric/geometric relationships can be used to determine characteristic values using the positions of the landmark locations and the associated lines/axes. In particular, for cases where the characteristic value is an angle, the angle, denoted as A (in radians) may be determined using 3 points of interest (e.g. 3 landmark locations) denoted as p1, p2, and p3, and can be calculated as the angle at p2 (i.e. the angle as measured at p2 when p2 is the vertex between a first line segment connecting p1 and p2 and a second line segment connecting p2 and p3) using Formula 1:

$$A = {cos}^{- 1} \frac{{s}_{1 - 2}^{2} + {s}_{2 - 3}^{2} - {s}_{1 - 3}^{2}}{2 \times {s}_{1 - 2} \times {s}_{2 - 3}} . \qquad Formula 1$$

In Formula 1, S_{a-b} is the length of the line segment between point a and point b, which can be calculated using Formula 2:

$${s}_{a - b} = \sqrt{{( {x}_{b} - {x}_{a} )}^{2} + {( {y}_{b} - {y}_{a} )}^{2}} . \qquad Formula 2$$

Additionally, the angle A between two lines (or axes), denoted as vectors v1 and v2, can be calculated using Formula 3:

$$A = a tan 2 ( ‖ {v}_{1} \times {v}_{2} ‖ , {v}_{1} \cdot {v}_{2} ) . \qquad Formula 3$$

Further, the distance d of a line between a line defined by two points p1 and p2, and a point p0 can be calculated using Formula 4:

$$d = \frac{\vert ( {x}_{2} - {x}_{2} ) \times ( {y}_{1} - {y}_{0} ) - ( {x}_{1} - {x}_{0} ) \times ( {y}_{2} - {y}_{1} ) \vert}{\sqrt{{( {x}_{2} - {x}_{1} )}^{2} + {( {y}_{2} - {y}_{1} )}^{2}}} . \qquad Formula 4$$

While Formulas 1-4 are applicable in at least some embodiments, other equations and relationships may also be used for the calculations of the characteristic values in different embodiments.

As depicted in FIG. 2, the servers 108 may utilize standard computational methods to determine the calculated characteristic values 124, which can be returned and/or displayed for further use.

Further, in addition to the above characteristic values, it is also possible to determine if the alignment of the lower limb is normal, varus or valgus, based on the calculated characteristic values. In particular, the value of the calculated HKA may be used to determine the lower limb alignment type. For example, where the HKA may be expressed as degrees of deviation from 180°, the alignment a particular limb may be classified as varus if the HKA is ≤—3°, valgus if the HKA is ≥3°, and normal otherwise. The lower limb alignment type may also be output as a characteristic value. More generally, the systems and methods of the present disclosure may be used to not just determine parameter values representative of pediatric lower limb alignment, but also to assess the alignment itself by comparing the one or more parameter values to a respective one or more thresholds indicative of normal alignment. When the parameter value(s) fall within those threshold(s), alignment is classified as normal; when the parameter value(s) fall outside those threshold(s), alignment is classified as abnormal.

In some embodiments, a third AI model 214 may be used to identify one or more anatomical regions of interest (212), for example, corresponding to entire bone structures. The third AI model 214 may be a CNN based AI model that is trained to identify one or more anatomical regions of interest. The training of the third AI model 214 is described in detail with reference to FIG. 5C and FIG. 6. The radiographic image 104 received by the servers 108 may be provided to the third AI model 120 as input. As such, the radiographic image may be resized to a certain standard to ensure consistency. Alternatively, zero padding scaling may be done to scale the radiographic image 104 to the standard image size.

The third AI model 214 can identify one or more anatomical regions of interest on the radiographic image 104, as shown in the highlighted regions of the processed radiographic image 212a of FIG. 3. As shown in FIG. 3, the third AI model 214 may be configured to identify entire bone structures or regions corresponding to entire bone structures. More generally, each anatomical region of interest can comprise a respective complete/entire bone and may identify the specific region in the radiographic image 104 that is occupied by the respective complete/entire bone. For example, the third AI model 214 may be configured to identify anatomical regions of interest corresponding to: the pelvis 304a, the (left and right) femur 304b, the (left and right) tibia 304c, the (left and right) fibula 304d, and the (left and right) foot 304e. The identified one or more anatomical regions of interest may be shown on a single radiographic image (e.g. the radiographic image 104) without any segmentation. Although FIG. 3 depicts the anatomical regions of interest as highlighted regions, the anatomical regions of interest may also be marked/identified in other ways such as by means of borders or within shapes (e.g. a box or oval). In some embodiments, the anatomical regions of interest may not be visually indicated, instead, data (e.g. coordinates in x and y) that would indicate the regions corresponding to the anatomical regions of interest may be output by the third AI model 214. In particular, each of the identified regions may be output as or identified using a mask. It should be noted that it is possible for anatomical regions to overlap with one another and as such, the anatomical regions of interest identified by the third AI model 214 may also overlap with one another.

As depicted in FIG. 2, the anatomical regions of interest identified by the third AI model 214 may be provided to the second AI model 122. The second AI model 122 may use the identified anatomical regions of interest in the determination of the anatomical features of interest. For example, the location/position/encompassed area of the identified anatomical regions of interest may be used by the second AI model 122 to improve the accuracy of feature identification. In some embodiments, the identified anatomical regions of interest may be used for the calculations of the characteristic values. In particular, the identified anatomical regions of interest can be used for the calculations of the physiological axes, the upper/lower joint lines of the knee, and/or the joint line of the foot, or to improve the accuracy thereof. For example, the identified anatomical region of interest corresponding to the femur may be used to more accurately determine the center of the femoral shaft such that the anatomical axis of the femur and the landmark location at the top of the greater trochanter can be more accurately identified. Similarly, the identified anatomical region of interest corresponding to the tibia may be used to more accurately determine the center of the tibial shaft such that the anatomical axis of the tibia can be more accurately identified. In some embodiments, outputs from the third AI model 214 can be used to identify the anatomical axes of the femur and tibia and the landmark location at the top of the greater trochanter.

According to a particular implementation of the present disclosure, the third AI model 214 may be a single CNN. The CNN may be residual network (ResNet) CNN and in particular may have 50 layers. The third AI model 214 may generate an augmented image comprising anatomical region(s) of interest corresponding to one or more of: the pelvis 304a, the (left and right) femur 304b, the (left and right) tibia 304c, the (left and right) fibula 304d, or the (left and right) foot 304e. The output image may be 512 pixels by 256 pixels, which is the same as the input radiographic image 104.

It should be noted that while the washer may not be an “anatomical” feature (of interest) in that it is not a part of the human body, it may still be processed by the first AI model 120 and the second AI model 122 in the same manner as other anatomical features of interest and therefore may be referred to as an anatomical feature of interest in the context of image processing and analysis by the first AI model 120 and the second AI model 122.

It should be noted that by using a combination of AI and algebraic computation (e.g. trigonometric calculations) to calculate the characteristic values, the overall computational burden and resource usage can be lower than that of an AI-only approach where the AI model(s) is configured to extract the characteristic values directly from radiographic images. In particular, by calculating the characteristic values using algebraic trigonometric relationships, the processes to be performed by the AI models may be limited to image processing, thereby reserving computational power for tasks that are particularly well suited for, and that benefit from, AI-enabled processing. That is, by determining the characteristic values using non-AI algebraic computation permits computational power to be focused on AI-enabled vision processing, thereby helping available computational power be used efficiently.

It should also be noted that the radiographic image 104 may include imaged hardware implants for patients with hardware implants in their lower limbs. The servers 108 can be configured to perform the above described process even for radiographic images where hardware implants are shown. In particular, the first, second, and third AI model 120, 122, and 214 may be trained such that they are able to perform image analysis and processing despite anomalies in artefacts present in the radiographic images that correspond to hardware implants, as described further with regard to FIGS. 5A-6.

FIG. 4 depicts a method 400 for automatic analysis of lower limb characteristics from radiographic images utilized by the process of FIG. 2. As depicted in FIG. 4, the radiographic image 104 of a patient/user may be taken (401) by equipment configured to capture radiographic images, such as an x-rays or computed tomography images. The captured radiographic image 104 can be stored for future access. The servers 108 may be configured to receive (402) the radiographic image 104. The radiographic image 104 may be transmitted from the capture equipment or received from a device 102 such as a computer. For example, a user may upload the radiographic image 104 to the servers 108 for analysis. The radiographic image 104 can be processed, for example, to be resized to a standard dimension that the servers 108 is configured to perform analysis for. The radiographic image 104 may also be processed by the servers to determine if the radiographic image 104 is of sufficient quality for image analysis. It should be noted that a GUI such one described with respect to FIG. 7 may be provided by the servers 108 for communication with the user for the exchange of data/information.

The servers 108 may receive one or more parameters (404), for example, from the user through the GUI. The parameters may be one or more characteristic values (as described in FIG. 2) for lower limb alignment from the radiographic image 104 that the user would like to determine. The parameters may also include conditions/criteria for the output results of the servers 108. For example, the user may also require the servers 108 to output intermediate processing products as parameters. Additionally, identified ROIs, anatomical features of interest, anatomical regions of interest, landmark locations, and segmented/highlighted radiographic image(s), which were described with reference to FIGS. 2 and 3, may be output. The user may also require the servers 108 to identify parameters such as the physiological axes, landmark locations, and other calculation criteria on the radiographic image 104. The servers 108 may also atomically calculate/output at least one or all of the above described parameters.

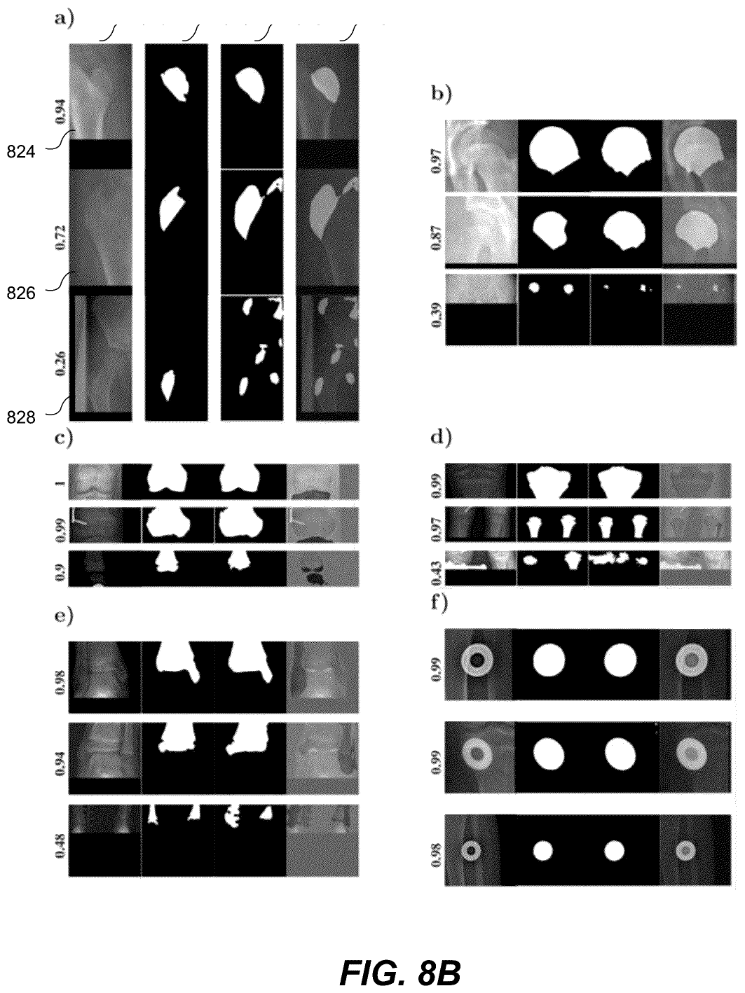

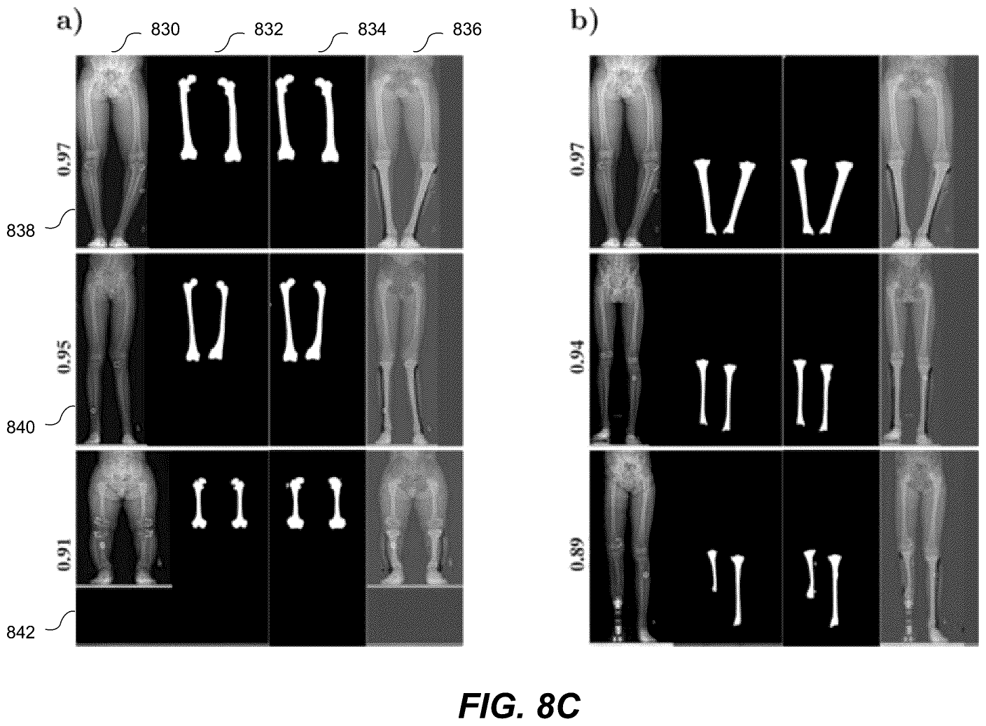

The radiographic image 104 is provided to the servers 108 for the identification of one or more ROIs (406). As described above with reference to FIGS. 2 and 3, one or more ROIs (e.g. 302a-302f) in the radiographic image 104a (for both limbs) may be identified, for example, by the first AI model 120 with the radiographic image 104 as the input. Each of the ROIs identified by the first AI model 120 may indicate or correspond to a particular anatomical feature of interest. For example, each ROI can identify a general location/region for a particular anatomical feature of interest, such as: femoral head, greater trochanter, distal femur, proximal tibia, distal tibia, or washer. Each of the identified ROIs may be output by the first AI model 120 as or using a mask corresponding to the region covered by the corresponding ROI or ROIs (408). In particular, each ROI may correspond to a specific mask. Alternatively, a single mask may be generated for multiple ROIs. Examples of masks generated by the first AI model 120 are shown and described with respect to FIG. 8a. Further, data representing the ROIs may also be output.

The first AI model 120 can segment (410) the radiographic image 104 to generate a plurality of segmented images (e.g. each being a cropped portion of the original radiographic image). The first AI model 120 may generate segmented images based on the identified ROIs such that each segmented image may correspond to or comprise a particular ROI or ROIs. For example, each segmented image may include ROI(s) that identifies one or more of: femoral head, greater trochanter, distal femur, proximal tibia, distal tibia, or washer.

The segmented images generated by the first AI model 120 may be provided to the second AI model 122. For example, the segmented images may be used as input images for the second AI model 122 to perform image analysis. As described above with references to FIGS. 2 and 3, one or more anatomical features of interest (e.g. 306a-306g) may be identified (412), for example, by the second AI model 122 from the segmented images (for both limbs). The second AI model 122 may be a CNN based AI model and can identify at least one anatomical feature from each segmented image directly or in combination with the corresponding ROIs identified by the first AI model 120. In particular, the at least one anatomical feature contained in each segmented image may be identified, which includes: femoral head, greater trochanter, distal femur, proximal tibia, distal tibia, fibula, foot (e.g. tibiotalar and/or talus), or washer. Each of the identified features may be output by the second AI model 122 as or using a mask corresponding to the region covered by the corresponding feature or features (414). In particular, each mask may correspond to a specific feature of interest. Alternatively, one or more masks may be generated, each corresponding to multiple features of interest. Examples of masks generated by the second AI model 122 are shown and described with respect to FIG. 8B. Further, segmented images where the anatomical features of interest are identified may also be output.

In accordance with the present disclosure, one or more landmark locations can be identified (416), for example by the second AI model 122, as described above with references to FIGS. 2 and 3. In particular, the identified one or more anatomical features of interest may be used as a basis to determine the one or more landmark locations. A landmark location (e.g. 308a-f) may be determined by the second AI model 122 from each segmented image based on the anatomical feature or features of interest in the corresponding segmented image. The identified landmark locations may correspond to: center of the femoral head, top of the greater trochanter corresponding to the center of the femoral shaft, upper knee center (e.g. on a line tangent to the femoral condyles), lower knee center (e.g. on a line tangent to the tibial plateau), center of the tibiotalar joint, and the center of the washer, where each of the landmark locations can be determined using the positions of the corresponding anatomical feature of interest or the positions of multiple anatomical features of interest. The locations (e.g. coordinates) of the identified landmark locations may be output.

The landmark locations determined by the second AI model 122 may be used to calculate (418) one or more characteristic values of lower limb alignment (for both limbs). In particular, as described above with reference to FIG. 2, by using the positions (e.g. coordinates) of the landmark locations, it is possible to determine/calculate/plot the physiological axes by relating the positions/coordinates of the landmark locations to each other. Specifically, trigonometric/geometric relationships can be used to determine characteristic values using the positions of the landmark locations and the associated axes (e.g. by using Formulas 1-4 or other trigonometric equations). The calculated characteristic values calculated may be include one or more of: mLPFA; mLDFA; mMPTA; mLDTA; aMPFA; aLDFA; aMPTA; aLDTA; aTFA; HKA; or MAD, which can be defined as described previously. Further, additional characteristic values corresponding to a type of lower limb alignment (e.g. normal, varus or valgus) can be classified using one or more calculated characteristic values (e.g. by using the HKA). The servers 108 may calculate the characteristic values selected by the user. Alternatively, some or all of the characteristic values may be calculated, for example, as determined by default or previously managed settings. The calculated characteristic values can be output as data or provided to the user (424). For example, the calculated values may be displayed on the GUI or as a file containing the calculated characteristic values as data.

In some embodiments, one or more anatomical regions of interest (e.g. entire bone structures) may be identified (420), for example, by the third AI model 214, as described above with references to FIGS. 2 and 3. The received radiographic image 104 (e.g. after processing) may be provided as input to the third AI model 214 such that the one or more anatomical regions of interest (e.g. 302a-e) in the radiographic image 104 may be identified on the radiographic image 104. For example, the anatomical regions of interest may respectively correspond to one or more of: the pelvis, the (left and right) femur, the (left and right) tibia, the (left and right) fibula, or the (left and right) foot. Further, the identified one or more anatomical regions of interest may be shown on a single radiographic image (e.g. the radiographic image 104) without any segmentation. Each of the identified anatomical regions of interest may be output by the third AI model 214 as or using a mask corresponding to the region covered by the corresponding anatomical region of interest (422). Examples of masks generated by the third AI model 214 are shown and described with respect to FIG. 8C. In some embodiments, mask(s) combining some or all of the identified anatomical regions of interest may be generated. Further, data representing the regions covered by anatomical regions of interest may also be output. The identified anatomical regions of interest may be used by the servers 108 for the calculations of the characteristic values (418), as shown in FIG. 4.

FIGS. 5A-5C respectively depict a process for training a first, second, and third AI model described in FIG. 2. It should be noted that the training process described herein may be repeatedly performed until a plateau accuracy of the AI models is reached.

FIG. 5A depicts a training process for the first AI model 120. The first AI model 120 may be a CNN-based AI model that is trained and configured to identify one or more ROIs corresponding to one or more features of interest. As depicted in FIG. 5A, training data can be captured/obtained (502a). The training data should be unmodified radiographic images from which characteristic values for lower limb alignment may be calculated. Specifically, the radiographic images may be anteroposterior standing weight-bearing radiographs. It should be noted that a large volume of training data may be required to train an AI model to accurately perform the designated task (e.g. identify ROIs). The training data may be obtained externally, for example, form a database of radiographic images (e.g. from a hospital). In some embodiments, the training data may be received from radiograph-taking instruments. For example, the radiographic images captured by one or more instruments over a period of time may be stored (504a) as training data, optionally in combination with externally obtained training data.

In accordance with the present disclosure, the training data may be processed (506a) before being provided to the first AI model 120 for training. The radiographic images may be resized or scaled to a certain standard to ensure consistency in training data. To train the first AI model 120 to identify the one or more ROIs, the radiographic images in the training data may be manually processed. In particular, each radiographic image may be processed to manually identify the one or more ROIs to be identified by the first AI model. The manually identified ROIs may be represented as masks (e.g. with “0” indicating absence and “1” indicating presence), which would be associated the corresponding radiographic images and provided to the first AI model 120 for training. To identify different ROIs, different masks may be manually produced. The manually produced masks may be used as a ground truth for training and testing. Examples of manually segmented masks used for the training of the first AI model 120 are shown in FIG. 8A. For example, the first AI model 120 may be trained to identify ROIs that identifies one of: femoral head, greater trochanter, distal femur, proximal tibia, distal tibia, and washer. Accordingly, for each radiographic image in the training data, a mask may be manually created for each ROI, where each ROI corresponds to an anatomical feature of interest being one of: femoral head, greater trochanter, distal femur, proximal tibia, distal tibia, and washer. As such, multiple masks may be created for a single radiographic image. In some embodiments, the first AI model 120 may comprise multiple algorithms such as CNNs. Each CNN may be trained to identify a particular ROI. In such cases, the training data may be categorized based on the identified ROI such that each CNN is only trained with training data for a specific ROI (e.g. a particular mask corresponding to one of the anatomical features of interest). To train the first AI model 120 to segment the radiographic image, the radiographic images in the training data may be manually segmented and provided to the first AI model 120 for training. The training data may be categorized based on the segmentation of the radiographic image such that each CNN is only trained to create a particular segmented image corresponding to one of the identified ROIs. To allow the first AI model 120 to identify ROIs for radiographic images that includes hardware implants, the training data may include radiographic images with hardware implants. These radiographic images may be processed to manually produce masks that identify the ROIs and to segment the radiographic image. The manually segmented images may be used as a ground truth for training and testing. By providing training data that includes the radiographic images with hardware implants as well as the corresponding masks/segmented images, the first AI model 120 may be trained to identify ROIs for radiographic images that contain hardware implants.

In some embodiments, the training data may be augmented to increase the volume of data available for training and to improve the accuracy of the trained model. For example, the processed training data (e.g. radiographic images) may be shifted randomly by a number of pixels vertically and/or horizontally, rotated a random number of degrees clockwise or counter clockwise, scaled up or down by a random factor, and/or reflected around the y-axis. It should be noted that the augmentation may be limited as to produce training data that is not unnatural (e.g. within the scope of a normal radiographic image). According to a particular embodiment, the training data augmentation may include: shifting up to 16 pixels horizontally and 32 pixels vertically and up to 32 pixels horizontally and 64 pixels vertically for a CNN trained to identify the ROI corresponding to the washer, rotating by up to 10 degrees clockwise or counter clockwise, and/or scaling up or down by a factor from 0.8 to 1.2.

The training data may be separated into a training set 508, validation set 510a, and testing set 512a and provided to the first AI model 120 for training (514a). The training process may be repeated to improve the accuracy of the trained model and/or to include further training data to improve the accuracy of the trained model. The trained first AI model 516a may be used by the server 108 as described above with respect to FIGS. 2 and 3.

According to a particular implementation, a total of 180 radiographic images can be processed and used as training data. For a first AI model 120 comprising 6 CNNs respectively configured to identify ROIs corresponding to femoral head, greater trochanter, distal femur, proximal tibia, distal tibia, and washer, the training set 508a includes 120 of the radiographic images while the validation set 510a and test set 512a include 30 of the radiographic images each. For the CNN trained to identify the washer, the respective number of radiographic images used for the training set 508a, validation set 510a, and testing set 512a may be 77, 20, and 30. The learning rate of the first AI model 120 may be 10-3 and the training process may be optimized using adaptive moment estimation (ADAM). The first AI model 120 may be trained over 20 epochs with a variable learning rate that is multiplied by 0.5 at 5 epoch intervals.

Referring now to FIG. 5B, a training process for the second AI model 122 is depicted. As shown in FIG. 5B, the process (i.e. 502b-516b) for training the second AI model 122 is substantially the same as the process (i.e. 502a-516a) depicted in FIG. 5a and described above with regard to the training of the first AI model 120. Notable differences between the training processes are described herein.

The second AI model 122 may be a CNN-based AI model that is trained and configured to identify one or more features of interest and one or more landmark locations. The training data can be captured/obtained (502b). The training data may the output data from the first AI model 120. Alternatively, the data may the training data (e.g. unmodified radiographic images) used for the training of the first AI model 120 or similar, manually processed for the training of the second AI model 122.

In accordance with the present disclosure, to train the second AI model 122 to identify the one or more anatomical features of interest, the output segmented images and identified ROIs from the first AI model 120 (e.g. once trained) may be provided for training. Alternatively, manually segmented images and identified ROIs may be provided for training. In particular, each segmented image comprising one or more ROIs may be processed to manually identify the one or more anatomical features of interest in the segmented image. The manually identified anatomical features of interest may be represented as masks, which would be associated with the corresponding segmented image and provided to the second AI model 122 for training. The identified ROIs may also be provided to the second AI model 122 for training (e.g. to improve the accuracy of feature identification). To identify different anatomical features of interest, different masks may be manually produced. The manually produced masks may be used as a ground truth for training and testing. Examples of manually segmented masks used for the training of the second AI model 122 are shown in FIG. 8B. For example, the second AI model 120 may be trained to identify anatomical features of interest corresponding to: femoral head, greater trochanter, distal femur, proximal tibia, distal tibia, fibula, foot (e.g. tibiotalar and/or talus), or washer. In some embodiments, mask(s) may be created to identify multiple anatomical features of interest. For each segmented image in the training data, a mask may be manually created for each anatomical feature of interest. In some embodiments, the second AI model 122 may comprise multiple neural networks such as CNNs. Each CNN may be trained to identify a particular anatomical feature of interest or particular anatomical features of interest. Specifically, each CNN may be trained to identify anatomical features of interest in a particular area (i.e. the segmented image). In such cases, the training data may be categorized based on the segmented images or the anatomical feature(s) of interest such that each CNN is only trained with training data for a specific image segment or anatomical feature(s) of interest (e.g. mask(s) corresponding to particular anatomical feature(s) of interest).

To train the second AI model 122 to identify the landmark locations, the radiographic images in the training data (e.g. segmented images) may be manually labeled or identified with the corresponding landmark location and provided to the second AI model 122 for training. For example, for each segmented image, a landmark location corresponding to one of: center of the femoral head, top of the greater trochanter corresponding to the center of the femoral shaft, upper knee center (e.g. on a line tangent to the femoral condyles), lower knee center (e.g. on a line tangent to the tibial plateau), center of the tibiotalar joint, or the center of the washer (depending on the position of the segmented image and the anatomical feature(s) of interest contained therein) may be manually identified (e.g. labelled using a coordinate in x and y), which can be provided to the second AI model 122 for training. The manually identified positions may be used as a ground truth for training and testing. The training data may be categorized based on the identified landmark location such that each CNN is only trained with training data for a specific landmark location (e.g. coordinates for a particular landmark location).

To allow the second AI model 122 to identify anatomical features of interest and landmark locations in radiographic images that include hardware implants, the training data does not exclude any radiographic image where hardware implants are shown and radiographic images with hardware implants are processed in the same manner as standard radiographic images. It should be noted that if the training data is received from the first AI model 120, the received training data can include data where hardware implants are present as the first AI model 120 may be trained with training data that includes hardware implants.

In some embodiments, the training data may be augmented to increase the volume of data available for training and to improve the accuracy of the trained model, as described above with reference to FIG. 5A. According to a particular embodiment, the training data augmentation may include: shifting up to 64 pixels horizontally and 64 pixels vertically, rotating by up to 30 degrees clockwise or counter clockwise, scaling up or down by a factor from 0.8 to 1.2, and/or reflect around the y-axis. For a second AI model comprising 6 CNNs respectively configured to identify anatomical features of interest in segmented images corresponding to femoral head, greater trochanter, distal femur, proximal tibia, distal tibia, and washer, the sizes of the training set 508b may respectively be: 244, 250, 151, 217, 221, 63, and 120, where the validation set 510b and test set 512b include 50 segmented radiographic images each. The learning rate of the second AI model 122 may be 10⁻² and the training process may be optimized using stochastic gradient descent with momentum (SGDM). The second AI model 122 may be trained over 100 epochs with a variable learning rate that is multiplied by 0.5 at 5 epoch intervals.

Referring now to FIG. 5C, a training process for the third AI model 214 is depicted. As shown in FIG. 5C, the process (i.e. 502c-516c) for training the third AI model 214 is substantially the same as the process (i.e. 502a-516a) depicted in FIG. 5A and described above with regard to the training of the first AI model 120. Notable differences between the training processes are described herein.

The third AI model 214 may be a CNN-based AI model that is trained and configured to identify one or more anatomical regions of interest. The training data for the third AI model 214 may be the same as the training data used for the training of the first AI model 120. Specifically, the training data may be unmodified radiographic images. To train the third AI model 214 to identify the one or more anatomical regions of interest, the radiographic images in the training data may be manually processed. In particular, each radiographic image may be processed to manually identify the one or more anatomical regions of interest to be identified by the third AI model 214. The manually identified anatomical regions of interest may be represented as masks and provided to the third AI model 214 for training. To identify different anatomical regions of interest, different masks may be manually produced. The manually produced masks may be used as a ground truth for training and testing. Examples of manually produced masks used for the training of the third AI model 214 are shown in FIG. 8C. For example, the third AI model 214 may be trained to identify anatomical regions of interest corresponding to one or more of: the pelvis, the (left and right) femur, the (left and right) tibia, the (left and right) fibula, or the (left and right) foot. Accordingly, for each radiographic image in the training data, a mask may be manually created for each anatomical region of interest. Alternatively, a mask for some or all of the anatomical regions of interest may be manually produced. In some embodiments, the third AI model 214 may comprise a single CNN configured to identify any number of the above identified anatomical regions of interest. To allow the third AI model 214 to identify anatomical regions of interest in radiographic images that include hardware implants, the training data may include radiographic images with hardware implants. These radiographic images can be processed to manually produce masks that identifies the anatomical regions of interest. By providing training data that includes the radiographic images with hardware as well as the corresponding masks, the third AI model 214 may be trained to identify anatomical region(s) of the interest for radiographic images that contain hardware implants.

In some embodiments, the training data may be augmented to increase the volume of data available for training and to improve the accuracy of the trained model, as described above with reference to FIG. 5A. According to a particular implementation, a third AI model comprising a CNN is trained using a training set 508c of 120 images, a validation set 510c of 30 images, and a testing set of 30 images. The learning rate of the second AI model 122 may be 10⁻² and the training process may be optimized using stochastic gradient descent with momentum (SGDM). The second AI model 122 may be trained over 100 epochs with a variable learning rate that is multiplied by 0.5 at 5 epoch intervals.

Referring now to FIG. 6, a method 600 for training AI model(s) used by the process of FIG. 2 is disclosed. As described above with reference to FIGS. 5A-5C, training data is received/obtained (602). The training data may be unmodified radiographic images of the lower limbs used to determine one or more characteristic values. The training data may be stored (604) in a training data database, for example in a case where the radiographic images are directly received from image capturing devices to provide a sufficient volume of training data. As shown in FIG. 6, 606a-614a, 606b-614b, and 606c-614c correspond to the training of the three different AI models 1-3. For example, the AI models may respectively be: the first AI model 120, the second AI model 122, and the third AI model 214. Specifically, AI model 1 may be trained to identify one or more ROIs corresponding to one or more features of interest and to segment the radiographic images to generate a plurality of segmented images each corresponding to a respective ROI; AI model 2 may be trained to identify one or more anatomical features of interest and one or more landmark locations; and AI model 3 may be trained to identify one or more anatomical regions of interest.

In accordance with the present disclosure, the training data may be processed (606a, 606b, 606c) prior to use in training. For example, the radiographic images may be resized or scaled to a certain standard to ensure consistency in the training data. The training data may also be augmented to increase the volume of training data.

For the training of AI model 1 (e.g. first AI model 120), the training data may be processed (606a) to manually identify the one or more ROIs to be identified by AI model 1, as described above with reference to FIG. 5A. To process the training data, mask(s) corresponding to the one or more ROIs may be created manually. For example, mask(s) corresponding to ROI(s) that identify one of: femoral head, greater trochanter, distal femur, proximal tibia, distal tibia, or washer may be created. In some embodiments, AI model 1 may comprise multiple neural networks such as CNNs where each CNN may be trained/configured to identify a particular ROI. In such cases, the training data may be categorized based on the identified ROI such that each CNN is only trained with training data for a specific ROI (e.g. a particular mask corresponding to one of the anatomical features of interest). The training data may also be processed to manually segment the radiographic image to create a plurality of segmented images, where each segmented image corresponds to a particular ROI. The training data may be categorized based on the segmentation of the radiographic image such that each CNN is only trained to create a particular segmented image corresponding to one of the identified ROIs. Processing of the training data to manually identify ROIs and to manually segment the radiographic image can also be performed for training data where hardware implants are included in the radiographic images such that AI model 1 can be trained to perform ROI identification and image segmentation on radiographic images with hardware implants.

For the training of AI model 2 (e.g. second AI model 122), the training data may be processed (606b) to manually identify the one or more anatomical features of interest and one or more landmark locations to be identified by AI model 2, as described above with reference to FIG. 5B. In particular, the training data to be provided to AI model 2 may be segmented images each with an identified ROI. The training data may be obtained from the radiographic images manually (e.g. manual ROI identification and image segmentation) or obtained from the output of a trained AI model 1, as shown in FIG. 6. To process the training data, masks corresponding to the one or more anatomical features may be created manually. For example, masks corresponding to one or more anatomical features of interest being one or more of: femoral head, greater trochanter, distal femur, proximal tibia, distal tibia, fibula, foot (e.g. tibiotalar and/or talus), or washer may be created. In some embodiments, masks may be created to identify multiple anatomical features of interest. For example, AI model 2 may be configured to identify anatomical feature(s) of interest in each segmented image. In some embodiments, AI model 2 may comprise multiple algorithms such as CNNs where each CNN may be trained/configured to identify a particular anatomical feature or features of interest. In such cases, the training data may be categorized based on the identified anatomical feature or features of interest such that each CNN is only trained with training data for a specific anatomical feature or features of interest (e.g. a particular mask corresponding to one of the anatomical feature(s) of interest). The training data may also be processed to manually identify one or more landmark locations, for example in each segmented image. The landmark locations identified may be one or more of: center of the femoral head, top of the greater trochanter corresponding to the center of the femoral shaft, upper knee center (e.g. on a line tangent to the femoral condyles), lower knee center (e.g. on a line tangent to the tibial plateau), center of the tibiotalar joint, or the center of the washer. The locations of the identified landmark locations may be provided for training as coordinates. The training data may be categorized based on the identified landmark locations such that each CNN is only trained with training data for a specific landmark location (e.g. coordinates for a particular landmark location). Processing of the training data to manually identify anatomical features of interest and to manually identify the landmark locations can also be performed for training data where hardware implants are included in the radiographic images such that AI model 2 can be trained to perform anatomical feature identification and landmark location identification on radiographic images with hardware implants.

For the training of AI model 3 (e.g. third AI model 214), the training data may be processed (606c) to manually identify the one or more anatomical regions of interest to be identified by AI model 3, as described above with reference to FIG. 5C. To process the training data, masks corresponding to one or more one or more anatomical regions of interest may be created manually. For example, masks corresponding to one or more anatomical regions being one or more of: the pelvis, the (left and right) femur, the (left and right) tibia, the (left and right) fibula, or the (left and right) foot may be created. In some embodiments, masks may be created to identify multiple (e.g. some or all) anatomical regions of interest. Processing of the training data to manually identify anatomical regions of interest can also be performed for training data where hardware implants are included in the radiographic images such that AI model 3 can be trained to perform anatomical region identification on radiographic images with hardware implants.

As depicted in FIG. 6, the processed training data can be provided to the respective AI model for training (608a, 608b, 608c). The processed training data may be divided into a training set, a validation set, and a testing set, which can be respectively used to train (610a, 610b, 610c), validate (612a, 612b, 612c), and test (614a, 614b, 614c) the three respective AI models. These processes may be repeated as more training data is gathered to improve the accuracy of the AI models. Once training is complete, the trained AI model 1 (616), trained AI model 2 (618), and trained AI model 3 (620) may be used by the servers 108 to perform calculations of characteristic values, as described above with reference to FIGS. 2 and 3.

FIG. 7 depicts a graphical user interface (GUI) 700 for the system of FIG. 1, according to an example embodiment. The GUI 700 may be configured for data input, data output, and data display, and may be communicatively coupled to the servers 108. As shown in FIG. 7, the GUI 700 may include a radiograph loading option 704, which can be used to retrieve a radiographic image from storage. The radiograph loading option 704 may also be configured to retrieve a plurality of radiographic images from storage such that batch image analysis can be performed by the servers 108 to return characteristic values for all of the retrieved radiographic images. A calculate button 706 can be provided on the GUI 700 to initialize image analysis as to calculate the characteristic values. Status indicators 708 may also be included in the GUI 700, which can be used to indicate that the image processing has completed (i.e. “Done”) or that an error has occurred and that image processing could not be completed (i.e. “ERROR”).

As depicted in FIG. 7, the uploaded radiographic image may be displayed in a radiograph display panel 702. Once the image processing/analysis is complete, the radiograph display panel 702 may be updated to show various characteristic features in the radiographic image. For example, the anatomical axes of the femur 716a and/or the anatomical axes of the tibia 716a may be shown, as depicted in FIG. 7. In some embodiments, the mechanical axes of the limb, femur, and/or tibia may be shown. Further, the (upper/lower) joint lines of the knee, joint lines of the foot, and/or the lines between the top of the greater trochanter corresponding to the center of the femoral shaft and the center of the femoral head may be shown. The radiograph display panel 702 may also show one or more markers 718 corresponding to the identified landmark locations or to show/track the bone structure.

The GUI 700 can also include axes plotting option 710, which can include an option to display (or not display) the calculated axes on the radiograph display panel 702. In some embodiments, the axes plotting option 710 can also include options to select the axes to be displayed. A washer size entry field 712 can be included on the GUI 700. By providing the size of the washer marker to the servers 108, it is possible to calculate characteristic values with respect to length, such as the MAD. The calculated characteristic values can be displayed on a results display 714. The displayed characteristic values may include values for both limbs and can include one or more of: mLPFA, mLDFA, mMPTA, mLDTA, aMPFA, aLDFA, aMPTA, aLDTA, aTFA, HKA, or MAD. Additional characteristic values may also be shown, such as the type of lower limb alignment (i.e. normal, varus, or valgus), which may be classified using previously calculated characteristic values such as HKA.

A working example of an embodiment of the present disclosure is described herein with respect to FIGS. 8A-13. In particular, FIGS. 8A-13 depict the effectiveness of the process of FIG. 2 by way of the working example. It should be noted that the analysis of the performance of the working example is performed with a particular set of radiographic images and are included purely as example performance results. It should also be noted that the working example is one particular implementation of the systems and methods of the present disclosure and that other implementations are possible as well. According to this particular implementation, the servers 108 are used to implement: a first AI model 120 comprising 6 CNNs respectively trained to identify 6 ROIs (i.e. corresponding to femoral head, greater trochanter, distal femur, proximal tibia, distal tibia, and washer) so as to generate 6 segmented images corresponding to the 6 ROIs; a second AI model 122 comprising 6 CNNs respectively trained to identify 6 anatomical features of interest (i.e. corresponding to femoral head, greater trochanter, distal femur, proximal tibia, distal tibia, and washer); and a third AI model 214 comprising a CNN trained to identify the femur, tibia and fibula. Landmark locations identified by the second AI model 122 can include: center of the femoral head, top of the greater trochanter corresponding to the center of the femoral shaft, upper knee center (e.g. on a line tangent to the femoral condyles), lower knee center (e.g. on a line tangent to the tibial plateau), center of the tibiotalar joint, or the center of the washer. Further details of the CNNs used for working example are provided below in Table 1 and Table 2.

TABLE 1
Working Example CNN Particulars

| | | Input | Output | |
|---|---|---|---|---|
| CNN Name | Architecture | Size | Size | Aim |
| FH box net | ResNet-50 | 512 × 256 | 256 × 256 | Femoral head ROI |
| GT box net | ResNet-50 | 512 × 256 | 512 × 256 | Greater trochanter |
| | | | | ROI |
| DF box net | ResNet-50 | 512 × 256 | 256 × 512 | Distal femur ROI |
| DT box net | ResNet-50 | 512 × 256 | 256 × 256 | Distal tibia ROI |
| PT box net | ResNet-50 | 512 × 256 | 256 × 512 | Proximal tibia ROI |
| Washer | ResNet-50 | 512 × 256 | 256 × 256 | Proximal tibia ROI |
| box net | | | | |
| FH net | ResNet-50 | 256 × 256 | 256 × 256 | Femoral head |
| GT net | ResNet-50 | 512 × 256 | 512 × 256 | Greater trochanter |
| DF net | ResNet-50 | 256 × 512 | 256 × 512 | Distal femur |
| DT net | ResNet-50 | 256 × 256 | 256 × 256 | Distal tibia |
| PT net | ResNet-50 | 256 × 512 | 256 × 512 | Proximal tibia |
| Washer net | ResNet-50 | 256 × 256 | 256 × 256 | Scale marker |
| | | | | (washer) |
| AA net | ResNet-50 | 512 × 256 | 512 × 256 | Femor/tibia/fibula |

TABLE 2
Working Example CNN Particulars

| CNN | Training | Validation | Testing | Learning | |
|---|---|---|---|---|---|
| Name | Set | Set | Set | Rate | Optimizer |
| FH box net | 120 | 30 | 30 | 10⁻³ | ADAM |
| OT box net | 120 | 30 | 30 | 10⁻³ | ADAM |
| DF box net | 120 | 30 | 30 | 10⁻³ | ADAM |
| DT box net | 120 | 30 | 30 | 10⁻³ | ADAM |
| PT box net | 120 | 30 | 30 | 10⁻³ | ADAM |
| Washer box | 77 | 30 | 30 | 10⁻³ | ADAM |
| net | | | | | |
| FH net | 244 | 50 | 50 | 10⁻² | SGDM |
| GT net | 250 | 50 | 50 | 10⁻² | SGDM |
| DF net | 151 | 50 | 50 | 10⁻² | SGDM |
| DT net | 217 | 50 | 50 | 10⁻² | SGDM |
| PT net | 221 | 50 | 50 | 10⁻² | SGDM |
| Washer net | 63 | 10 | 10 | 10⁻² | SGDM |
| AA net | 120 | 30 | 30 | 10⁻² | SGDM |

ADAM: adaptive moment estimation
SGDM: stochastic gradient descent with momentum

Statistical analysis was performed to measure the accuracy and effectiveness of the working example. Accuracy values disclosed herein describe the proportion of pixels in an image that are correctly labeled and range from 0 (0% correct) to 1 (100% correct). Sørensen-Dice similarity coefficients, also referred to as Dice scores, are used herein to evaluate performance. For two sets of data (A and B), such as results obtained manually and results obtained via the working example, the Dice score (D) may be calculated using Formula 5 and Formula 6, provided below.

$$D ( A , B ) = 2 \times \frac{\vert A ⋂ B \vert}{\vert A \vert + \vert B \vert} \qquad Formula 5$$

$$D ( A , B ) = 2 \times \frac{2 \times TP}{2 \times TP + FP + FN} \qquad Formula 6$$

Referring now to FIG. 8A, the ROIs identified by the working example are compared to the ROIs identified manually by a orthopedic surgery fellow. The ROI identification and image segmentation as shown in FIG. 8A may be performed by the first AI model 120. In each set of images, the original (input) radiographic image 804, the manually identified ROIs 806, the ROIs identified by the working example 808, and the segmented images 810 generated by the working example are provided. Further, for each set of images, the top row of images 812 and the bottom row of images 814 respectively represent the results with the best Dice score and the results with the worst Dice score (shown to the left of the row of images). It should be noted that each set of images corresponds to the outputs of a particular CNN of the first AI model 120 and particularly relate to an ROI corresponding to one of: femoral head, greater trochanter, distal femur, proximal tibia, distal tibia, or washer. As depicted in FIG. 8A, the ROIs identified by the working example (808) and identified manually (806) are shown as masks. The segmented images 810 may be segmented based on the ROIs identified by the working example 806. Further, as shown in segmented image 810a, the working example is able to identify ROIs and accordingly generate segmented images for radiographic images that include hardware implants.

Referring now to FIG. 8B, the anatomical features of interest identified by the working example is compared to the anatomical features of interest identified manually by a orthopedic surgery fellow. The identification of the anatomical features of interest as shown in FIG. 8B may be performed by the second AI model 122. In each set of images, the (input) segmented image 816, the manually identified anatomical features of interest 818, the anatomical features of interest identified by the working example 820, and the final segmented images 822 generated by the working example showing all identified anatomical features of interest are provided. Further, for each set of images, the top row of images 824, middle row of images 826, and the bottom row of images 828 respectively represent the results with the best Dice score, the mean Dice score and the worst Dice score (shown to the left of the row of images). It should be noted that each set of images corresponds to the outputs of a particular CNN of the second AI model 122 and particularly relate to an anatomical feature of interest corresponding to one of: femoral head, greater trochanter, distal femur, proximal tibia, distal tibia, or washer. As depicted in FIG. 8B, the anatomical features of interest identified by the working example (820) and identified manually (818) are shown as masks. It should be noted that for certain regions (e.g. segmented images), multiple anatomical features of interest may be identified to generate the final segmented images 822, however, only one mask corresponding to one of the identified anatomical features of interest is shown.

Referring now to FIG. 8C, the anatomical regions of interest identified by the working example is compared to the anatomical regions of interest identified manually by an orthopedic surgery fellow. The identification of the anatomical regions of interest as shown in FIG. 8C may be performed by the third AI model 214. In each set of images, the original (input) radiographic image 830, the manually identified anatomical regions of interest 832, the anatomical regions of interest identified by the working example 834, and the final radiographic image 836 generated by the working example showing all identified anatomical regions of interest are provided. Further, for each set of images, the top row of images 838, middle row of images 840, and the bottom row of images 842 respectively represent the results with the best Dice score, the mean Dice score and the worst Dice score (shown to the left of the row of images). As depicted in FIG. 8C, the anatomical features of interest identified by the working example (834) and identified manually (832) are shown as masks. It should be noted that multiple anatomical regions of interest may be identified to generate the final radiographic images 836, however, only one mask corresponding to one of the identified anatomical features of interest is shown. The identified anatomical regions of interest shown in the final radiographic images 836 include the femur, tibia, and fibula.

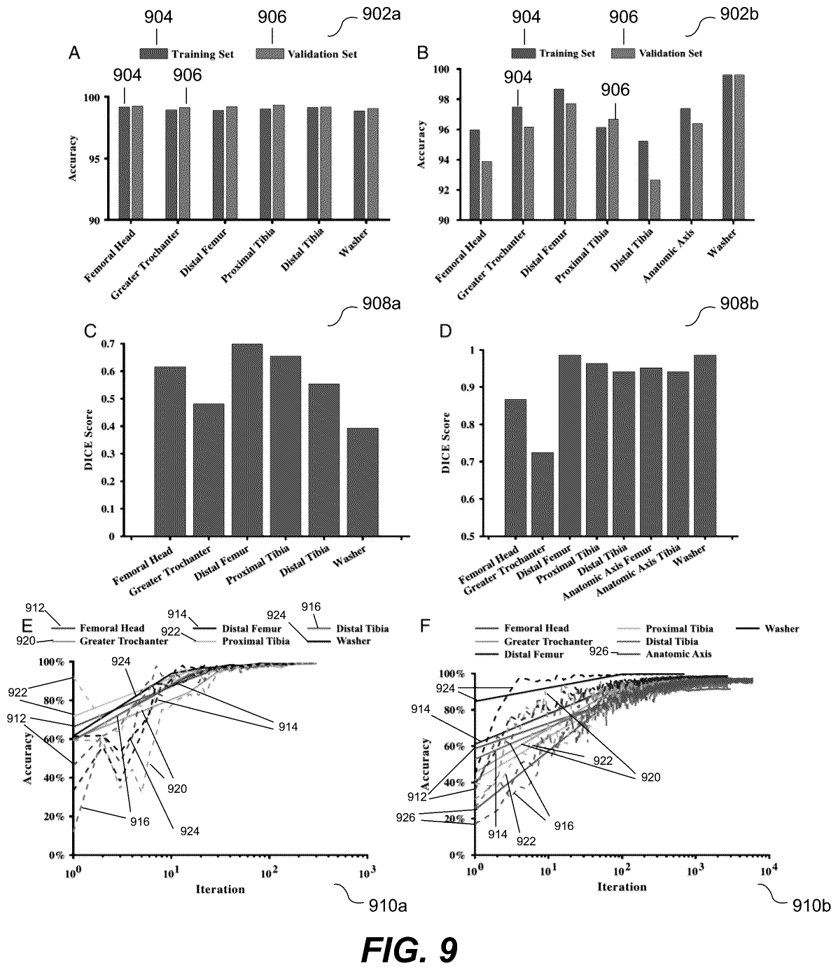

FIG. 9 depicts the performance of the working example in identifying ROIs and anatomical features of interest. The identified ROIs and anatomical features of interest correspond to the femoral head, greater trochanter, distal femur, proximal tibia, distal tibia, and washer. The ROI identification may be performed by the first AI model 120 while the anatomical feature of interest identification may be performed by the second AI model 122. Accuracy charts 902a and 902b respectively depict the accuracy of the working example in identifying ROIs and anatomical features of interest (e.g. once the AI models are fully trained). Results for the training set (904) and results for the validation set (906) are shown. As depicted in FIG. 9, the accuracy of AI models in identifying ROIs and anatomical features of interest correspond to the femoral head, greater trochanter, distal femur, proximal tibia, distal tibia, and washer are above 90%. Dice score charts 908a and 908b respectively depict the Dice scores of the working example in identifying ROIs and anatomical features of interest (e.g. once the AI models are fully trained). As depicted in FIG. 9, relatively high Dice scores are achieved for the identification of the anatomical features of interest. Model training accuracy graphs 910a and 910b respectively depict the accuracy of the working example in identifying ROIs and anatomical features of interest over the course of training for the respective AI model. One iteration may be classified as training with 8 images. The performance of the working example in identifying ROIs and anatomical features of interest corresponding to femoral head (912), greater trochanter (920), distal femur (914), proximal tibia (922), distal tibia (916), and washer (924) are shown in model training accuracy graphs 910a and 910b. The performance of the working example in identifying the anatomic axis (926) is also shown in accuracy graph 910b. The solid lines refer to performances for the training set and the dashed lines refer to the performances for the validation set.

FIG. 10 depicts the analysis of the characteristic values calculated by the working example. In comparison graphs 1002, the MAD, mLDFA, and mMPTA values calculated by the working example (y) are respectively plotted against the same values measured manually by an orthopedic surgery fellow (x). As shown in the comparison graphs 1002, the MAD, mLDFA, and mMPTA values have good agreement between the calculated and measured values. Distribution graphs 1004 depict the distribution of calculated MAD, mLDFA, and mMPTA values. It should be noted that the calculated values are normally distributed. In scatter plots 1005, the differences between the calculated MAD, mLDFA, and mMPTA values and the manually measured MAD, mLDFA, and mMPTA values (y) are respectively plotted against the manually measured MAD, mLDFA, and mMPTA values (x). As depicted in FIG. 10, the differences are not a function of the measured values, which can indicate that the baseline level of error is not exacerbated with more extreme values associated with surgical deformity.

Summary statistics for the performance of the working example in calculating MAD, mLDFA, and mMPTA values compared to (e.g. differences) manual calculations by an orthopedic surgery fellow are shown below in Table 3.

TABLE 3
Working Example Summary Statistics

| Measure- | | | | | | Gaussian Parameters | | |
|---|---|---|---|---|---|---|---|---|
| ment | Mean | Median | STD | Q10 | Q90 | μ | σ | R³ |
| MAD | 2.02 | 1.52 | 2.04 | 0.30 | 4.23 | 0.23 | 3.27 | 0.97 |
| LDFA | 1.73 | 1.00 | 3.14 | 0.20 | 3.20 | −0.05 | 1.90 | 0.99 |
| MPTA | 2.90 | 2.60 | 2.04 | 0.50 | 5.20 | −2.02 | 4.14 | 0.92 |

STD: standard deviation
Q: quantile

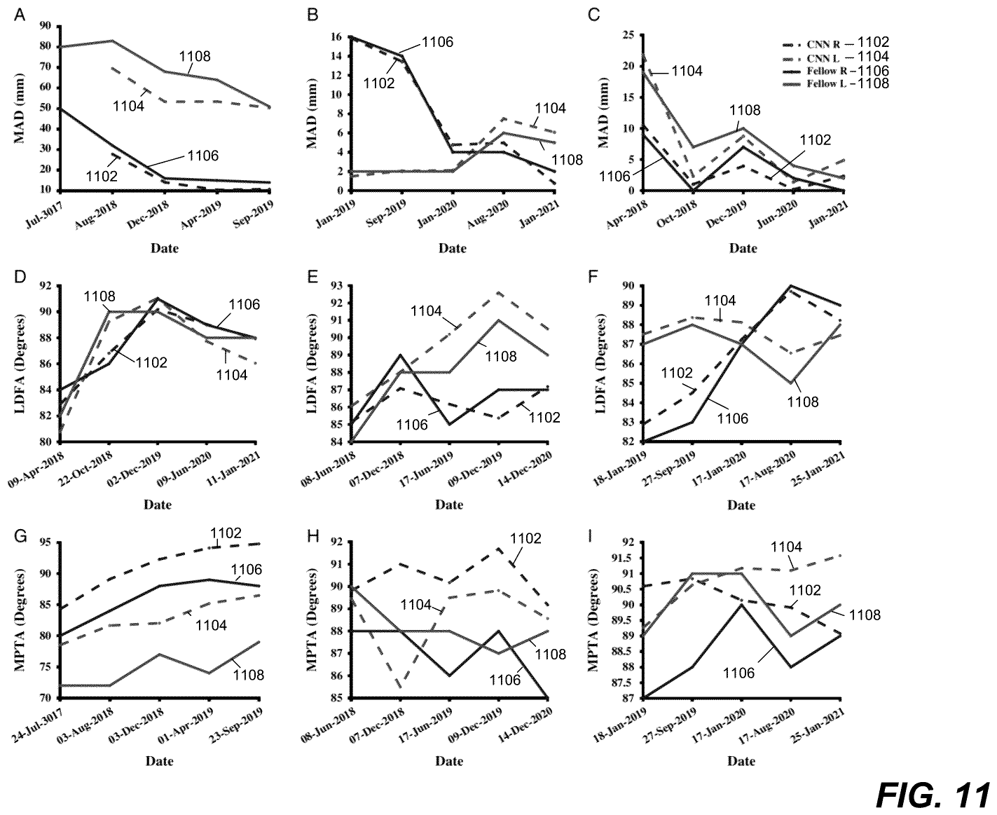

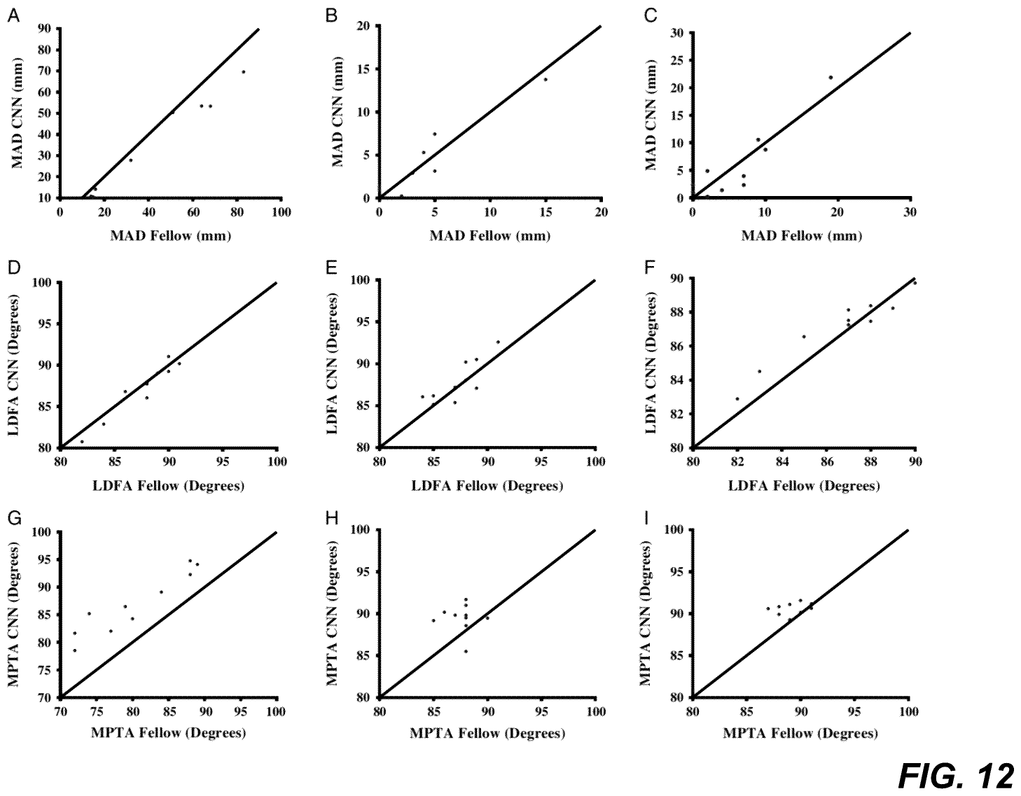

FIGS. 11 and 12 depict the lower limb characteristic values determined by the process of FIG. 2 for the working example in comparison to the same lower limb characteristic values determined manually. FIGS. 11 and 12 depict the MAD, mLDFA, and mMPTA values calculated from a series of radiographic images over time (e.g. the radiographic images captured over the course of multiple hospital visits) for three patients as determined by the process of FIG. 2 and the MAD, mLDFA, and mMPTA values for the same series of radiographic images as measured manually by an orthopedic surgery fellow. It should be noted that each patent's radiographic images were studied by the same orthopedic surgery fellow. In FIG. 11, the capture dates of the radiographic images are plotted on the x-axis while the characteristic values are plotted on the y-axis. On each graph depicted in FIG. 11, the changes in the characteristic values over time are shown using individual trend-lines. Specifically, each graph includes four trend-lines in which: a first trend-line 1102 corresponds to the characteristic values for the right lower limb as determined by the process of FIG. 2, a second trend-line 1104 corresponds to the characteristic values for the left lower limb as determined by the process of FIG. 2, a third trend-line 1106 corresponds to the characteristic values for the right lower limb as measured by the orthopedic surgery fellow, and a fourth trend-line 1108 corresponds to the characteristic values for the left lower limb as measured by the orthopedic surgery fellow. As shown in FIG. 11, the characteristic values calculated by the working example show good agreement in measurements and overall trend when compared to the same values measured by the orthopedic surgery fellow, which can demonstrate that the disclosed systems and methods are sufficiently accurate in the automatic calculations of the characteristic values.

Similarly, FIG. 12 depicts the lower limb characteristic values determined by the process of FIG. 2 for the working example plotted against the same lower limb characteristic values determined manually. As depicted in FIG. 12, characteristic values determined by the process of FIG. 2 are plotted as y-values and the characteristic values for measured manually are plotted as x-values. The diagonal line in each graph represents a perfect match (i.e. where the x-value is equal to the y-value) between the automatically and manually calculated values. As shown in FIG. 12, the values calculated by the working example for the three patients largely corresponds to the values measured by the a orthopedic surgery fellow, which can demonstrate that the disclosed systems and methods are sufficiently accurate in the automatic calculations of the characteristic values.

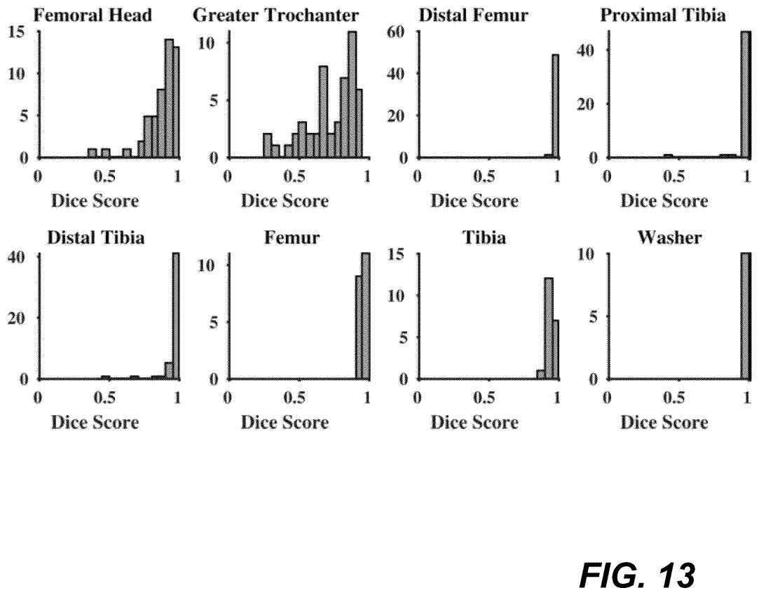

Referring now to FIG. 13, the distributions of performance scores for example lower limb anatomical features determined by the process of FIG. 2 are depicted. As described above, for the working example, the process as described in FIG. 2 can identify features corresponding to the femoral head, greater trochanter, distal demur, proximal tibia, distal tibia, (entire) femur, (entire) tibia, and washer. For each feature, the accuracy of the automatic determination is compared against manual determination using Dice scores, with the distribution of Dice scores for each feature shown in a respective graph depicted in FIG. 13. As shown in the graphs of FIG. 13 where the frequencies are plotted on the y-axis and Dice scores are plotted on the x-axis, the distribution of Dice score for all features are skewed towards 1, indicating good accuracy for the disclosed process. The identification of distal demur, proximal tibia, distal tibia, (entire) femur, (entire) tibia, and washer for the working example is particularly accurate, as shown by the heavy skew of the Dice score distribution to the value of 1. In comparison, the femoral head and the greater trochanter are identified in the working example with less accuracy, which reduces the mean Dice scores of the disclosed process. It is possible that the reduction in accuracy may be the result of the AI models analyzing images with low resolution and where certain anatomy feature(s) are obstructed, thereby reducing the ability of the AI model to interpret where the feature is located.

It would be appreciated by one of ordinary skill in the art that the system and components shown in the figures may include components not shown in the drawings. For simplicity and clarity of the illustration, elements in the figures are not necessarily to scale and are only schematic. It will be apparent to persons skilled in the art that a number of variations and modifications can be made without departing from the scope of the invention as described herein.

It is contemplated that any part of any aspect or embodiment discussed in this specification can be implemented or combined with any part of any other aspect or embodiment discussed in this specification, so long as such those parts are not mutually exclusive with each other.

It should be recognized that features and aspects of the various examples provided above can be combined into further examples that also fall within the scope of the present disclosure.

When used in this specification and claims, the terms “comprises” and “comprising” and variations thereof mean that the specified features, steps or integers are included. The terms are not to be interpreted to exclude the presence of other features, steps or components. Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections. Further, the singular forms “a”, “an”, and “the” are intended to include the plural forms as well, unless the context clearly indicates otherwise.

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

Use of language such as “at least one of X, Y, and Z,” “at least one of X, Y, or Z,” “at least one or more of X, Y, and Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

1. A pediatric lower limb alignment assessment method, the method comprising:
receiving a pediatric lower limb radiographic image;
identifying a plurality of regions of interest (ROIs) in the radiographic image using a first artificial intelligence (AI) model, each one of the plurality of ROIs containing at least one of a plurality of anatomical features of interest, each anatomical feature of interest comprising a respective portion of a bone;
determining a plurality of landmark locations for each one of the plurality of identified ROIs in the radiographic image using a second AI model, each landmark location corresponding to a position within a respective anatomical feature of interest; and
calculating at least one parameter value representative of the pediatric lower limb alignment based on a geometric relationship between the plurality of landmark locations.

2. The method of claim 1, further comprising:
segmenting the radiographic image to generate a plurality of image segments using the first AI model, each one of the plurality of image segments corresponding to one of the plurality of ROIs,
wherein each one of the plurality of landmark locations is determined from a respective one of the plurality of image segments by the second AI model.

3. The method of claim 1, further comprising:
capturing the radiographic image.

4. The method of claim 1, further comprising:
identifying a plurality of anatomical regions of interest using a third AI model, each anatomical region of interest comprising an entire bone; and
determining the plurality of landmark locations using the plurality of anatomical features of interest and the plurality of anatomical regions of interest.

5. The method of claim 1,
wherein the second AI model is configured to identify the plurality of anatomical features of interest; and
wherein the plurality of landmark locations are determined using the plurality of anatomical features of interest.

6. The method of claim 1, wherein the radiographic image is an anteroposterior standing weight-bearing radiograph.

7. The method of claim 1, wherein the radiographic image includes hardware implants.

8. The method of claim 1, further comprising:
obtaining radiographic images where at least one region of interest is identified; and
training the first AI model using the obtained radiographic images to identify the at least one identified region of interest.

9. The method of claim 1, further comprising:
obtaining image segments where each image segment corresponds to a respective region of interest; and
training the first AI model using the obtained image segments to segment the radiographic image to generate a plurality of image segments based on the plurality of ROIs.

10. The method of claim 1, further comprising:
obtaining radiographic images where at least one anatomical feature of interest is identified; and
training the second AI model using the obtained radiographic images to identify the at least one identified anatomical feature of interest.

11. The method of claim 1, further comprising:
obtaining radiographic images where at least one landmark location is identified; and
training the second AI model using the obtained radiographic images to identify the at least one identified landmark location.

12. The method of claim 2, further comprising;
obtaining radiographic images where at least one anatomical region of interest is identified; and
training the third AI model using the obtained radiographic images to identify the at least one identified anatomical region of interest.

13. The method of claim 1, wherein the plurality of ROIs and the plurality of anatomical features of interest comprise regions corresponding to: femoral head, greater trochanter, distal femur, proximal tibia, distal tibia, or combinations thereof.

14. The method of claim 1, wherein the plurality of ROIs and the plurality of anatomical features of interest comprise a region corresponding to a radiopaque washer used as a size marker.

15. The method of claim 1, wherein the first AI model and/or the second AI model is a residual neural network.

16. The method of claim 1,
wherein the first AI model comprises five convolutional neural networks (CNNs), each configured to identify a respective region of interest corresponding to one of: femoral head, greater trochanter, distal femur, proximal tibia, and distal tibia; and
wherein the first AI model comprises an additional convolutional neural network configured to identify a region of interest corresponding to a washer.

17. The method of claim 1,
wherein the second AI model comprises five convolutional neural networks (CNNs), each configured to identify a respective anatomical feature of interest corresponding to one of: femoral head, greater trochanter, distal femur, proximal tibia, and distal tibia; and
wherein the second AI model comprises an additional convolutional neural network configured to identify a feature of interest corresponding to a washer.

18. The method of claim 1, wherein the at least one parameter value is at least one of: mechanical axes of the femur and tibia; a hip-knee angle; a mechanical lateral proximal femoral angle; a mechanical lateral distal femoral angle; a mechanical medial proximal tibial angle; a mechanical lateral distal tibial angle; a mechanical axis deviation; an anatomic medial proximal femoral angle; an anatomic lateral distal femora angle; an anatomic medial proximal tibial angle; an anatomic lateral distal tibial angle; an anatomic tibiofemoral angle; or a knee alignment.

19. A system for determining a parameter value of lower limb alignment, the system comprising one or more processing units configured to perform pediatric lower limb alignment assessment method, the method comprising:
receiving a pediatric lower limb radiographic image;
identifying a plurality of regions of interest (ROIs) in the radiographic image using a first artificial intelligence (AI) model, each one of the plurality of ROIs containing at least one of a plurality of anatomical features of interest, each anatomical feature of interest comprising a respective portion of a bone;
determining a plurality of landmark locations for each one of the plurality of identified ROIs in the radiographic image using a second AI model, each landmark location corresponding to a position within a respective anatomical feature of interest; and
calculating at least one parameter value representative of the pediatric lower limb alignment based on a geometric relationship between the plurality of landmark locations.

20. A non-transitory computer-readable medium having computer readable instructions stored thereon, which, when executed by one or more processing units, causes the one or more processing units to perform a pediatric lower limb alignment assessment method, the method comprising:
receiving a pediatric lower limb radiographic image;
identifying a plurality of regions of interest (ROIs) in the radiographic image using a first artificial intelligence (AI) model, each one of the plurality of ROIs containing at least one of a plurality of anatomical features of interest, each anatomical feature of interest comprising a respective portion of a bone;
determining a plurality of landmark locations for each one of the plurality of identified ROIs in the radiographic image using a second AI model, each landmark location corresponding to a position within a respective anatomical feature of interest; and
calculating at least one parameter value representative of the pediatric lower limb alignment based on a geometric relationship between the plurality of landmark locations.
